(19) United States
(12) Patent Application Publication
(10) Pub. No.: US 2025/0108129 A1
(43) Pub. Date: Apr. 3, 2025

(54) Exon skipping to treat Usher Syndrome

(71) Applicant: Massachusetts Eye and Ear Infirmary, Boston, MA (US)

(72) Inventors: Zheng-Yi Chen, Chestnut Hill, MA (US); Wenliang Zhu, Malden, MA (US)

(21) Appl. No.: 18/833,060

(22) PCT Filed: Feb. 3, 2023
(86) PCT No.: PCT/US2023/012284

§ 371 Date: Jul. 25, 2024

Related U.S. Application Data
(60) Provisional application No. 63/306,833, filed on Feb. 4, 2022.

(51) Int. Cl. A61K 48/00; A61P 27/16; C12N 9/22; C12N 15/11; C12N 15/86

(52) U.S. Cl. CPC A61K 48/005; A61P 27/16; C12N 9/22; C12N 15/111; C12N 15/86; C12N 2310/20; C12N 2320/33; C12N 2750/14143

(57) ABSTRACT

Compositions for use in treating subjects with USH2A-associated retinal and/or cochlear disease that result from mutations in exon 13 of the USH2A gene by deletion of exon 13 splicing acceptor sequences from the USH2A gene or transcripts, and methods of use thereof, as well as genetically modified animals and cells.

### CROSS-REFERENCE TO RELATED APPLICATIONS

This application claims the benefit of U.S. Provisional Patent Application No. 63/306,833, filed Feb. 4, 2022. The contents of this application are incorporated herein by reference in its entirety.

### FEDERALLY SPONSORED RESEARCH OR DEVELOPMENT

This invention was made with Government support under Grant Nos. DC016875 and TR002636 awarded by the National Institutes of Health. The Government has certain rights in the invention.

### TECHNICAL FIELD

Compositions for use in treating subjects with USH2A-associated retinal and/or cochlear degeneration that result from mutations in exon 13 of the USH2A gene by deletion of exon 13 splicing acceptor sequences from the USH2A gene or transcripts, and methods of use thereof, as well as genetically modified animals and cells.

### BACKGROUND

Usher syndrome (USH) is the leading cause of inherited combined hearing and vision loss. Among patients with Usher syndrome, approximately two out of three suffer from Usher syndrome type II (USH2), of whom more than 75% of cases are caused by mutations in the USH2A gene. Patients with USH2 display moderate-to-severe hearing loss, post-pubertal onset of retinitis pigmentosa (RP) and normal vestibular reflexes, and USH2 represents the most common form of inherited deaf-blindness and is estimated to affect approximately 1 in 17,000 individuals. In particular, mutations in exon 13 account for approximately 35% of all USH2 cases, including a single base deletion at position 2299 (c.2299delG), which is the most common mutation accounting for about 24% of cases.

### SUMMARY

Provided herein are nucleic acids comprising sequences encoding a Cas9 protein, and a first gRNA, and a second gRNA, wherein the target sequence of the first gRNA is any one of SEQ ID NOs: 2-244 provided in Table 1, and the target sequence of the second gRNA is any one of SEQ ID NOs: 245-431 provided in Table 2. The target sequence of the first gRNA falls in the 3′ 1000 base-pairs (bp) of intron 12 and the target sequence of the second gRNA falls in exon 13; any combination of a first gRNA having a target sequence of any one of SEQ ID NOs: 2-244, and a second gRNA having a target sequence of any one of SEQ ID NOs: 245-431 can be used, in order to delete the relevant DNA fragment from the genome. In some embodiments, the target sequence of the first gRNA is SEQ ID NO: 17 and the target sequence of the second gRNA is SEQ ID NO: 313. In some embodiments, the target sequence of the first gRNA is SEQ ID NO: 17 and the target sequence of the second gRNA is SEQ ID NO: 260. A deletion mediated by paired-gRNAs provided herein will include part of intron 12 and part of exon 13, with deletion size ranging from 6 bp to several kilobase-pairs (kb), all of which can induce human USH2A exon 13 skipping, with the preferred range being 6 kb to 2 kb, with certain preferred combinations of gRNAs.

In some embodiments, the nucleic acid encodes S. pyogenes Cas9 or S. aureus Cas9 (optionally KKH SaCas9). In some embodiments, the Cas9 comprises a nuclear localization signal, e.g., a C-terminal nuclear localization signal and/or an N-terminal nuclear localization signal; and/or wherein the sequences encoding Cas9 comprises a polyadenylation signal.

In some embodiments, the gRNA is a unimolecular S. aureus or S. pyogenes gRNA, or the corresponding two-part modular S. aureus or S. pyogenes gRNA (see, e.g., WO 2018/026976).

In some embodiments, the nucleic acid comprises a viral delivery vector, preferably an adeno-associated virus (AAV) vector. In some embodiments, the viral delivery vector comprises a promoter for Cas9, preferably a CMV, EFS, U1A or hGRK1 promoter. In some embodiments, the nucleic acid comprises:
(i) a first guide RNA comprising a targeting domain sequence selected from any one of SEQ ID NOs: 2-244 and a second guide RNA comprising a targeting domain sequence selected from any one of SEQ ID NOs: 245-431;
(ii) a first and a second inverted terminal repeat sequence (ITR); and
(iii) a promoter for driving expression of the Cas9 selected from the group consisting of a CMV, an EFS, U1A or an hGRK1 promoter.

The nucleic acids described herein can be used, e.g., in therapy, or in preparation of a medicament. For example, the nucleic acids can be uses in a method of treating a subject who has a condition associated with a mutation in exon 13 of the USH2A gene.

In some embodiments, the condition is Usher Syndrome type 2 or autosomal recessive retinitis pigmentosa (arRP).

In some embodiments, the AAV vector is delivered to a retina of a subject by injection, such as by subretinal injection, or is delivered to the inner ear of a subject by injection, e.g., through the round window.

Additionally provided herein are compositions comprising first ribonucleoprotein (RNP) complexes comprising a Cas9 protein and a first gRNA or sgRNA, and/or second RNP complexes comprising a Cas9 protein and a second gRNA or sgRNA, wherein the target sequence of the first gRNA is any one of SEQ ID NOs: 2-244 and the target sequence of the second gRNA is any one of SEQ ID NOs: 245-431. In some embodiments, the Cas9 is S. aureus Cas9, optionally KKH SaCas9 (optionally KKH SaCas9) or S. pyogenes Cas9.

Further provided are methods for deleting a sequence comprising an exon 13 splicing acceptor sequence from the USH2A gene in a cell, wherein the deletion contains part of intron 12 and part of exon 13, ranging from 6 bp to several kb, all of which could induce human USH2A exon 13 skipping, though the ideal range is below 2 kb. The methods include contacting the cell with a nucleic acid or composition as described herein.

Also provided herein are methods for genome editing in human cells, including using CRISPR editing to form a first double strand break within intron 12 of the human USH2A gene and a second double strand break within exon 13 of the human USH2A gene and results in the removal of a fragment of genome DNA containing part of intron 12 and part of exon 13 of the USH2A gene on chromosome 1. In some embodiments, the first double strand break is generated using a gRNA having a target sequence of any one of SEQ ID NOs: 2-244 and the second double strand break is generated using a gRNA having a target sequence of any one of SEQ ID NOs: 245-431.

In some embodiments, the cell is in or from a subject who has a mutation in the USH2A gene. In some embodiments, the cell is a cell of the eye or inner ear of a mammal.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1. Schematic diagram of CRISPR/Cas9 splicing acceptor targeting strategy to induce USH2A exon 13 skipping by a pair of gRNAs targeting the flanking genomic DNA of the splicing acceptor site.

FIGS. 2A-I. Splicing acceptor targeting strategy increases exon 12 (human exon 13 equivalent) skipping in-frame Ush2A transcript in mouse cells.
(a). Gel image showing full exon 12 deletion strategy in the genome using CRISPR/Cas9. Red asterisks indicate fragment after deletion.
(b). Schematic diagram of full exon 12 deletion strategy.
(c). Next generation sequencing (NGS) of edited genomic DNA confirming the deletion of exon 12.
(d). Gel image showing splicing acceptor targeting strategy deleting Ush2A exon 12 splicing acceptor in HEI-OC1 cells. Asterisk indicates the fragment after deletion.
(e). Schematic diagram of splicing acceptor targeting strategy.
(f). NGS demonstrating genome editing efficiency of full exon 12 deletion and splicing acceptor targeting deletion strategy.
(g). Quantification of genome editing efficiency of full exon 12 deletion (sgL1+sgR1) and splicing acceptor targeting (sgL1+sgK4) deletion strategy.
(h). Gel image showing different Ush2A transcripts with or without genome editing.
(i). qPCR quantification of exon 12-unskipped (upper) and -skipped (lower) transcripts with or without genome editing.

FIGS. 3A-H. Efficient genome editing at USH2A exon 13 loci using splicing acceptor targeting strategy in human cells.
(a). Schematic diagram of full exon 13 deletion strategy shows expected in-frame transcript with the skipped exon13.
(b). Gel image showing full USH2A exon 13 deletion in WERI-RB1 cells genome. Asterisks indicate fragments after deletion.
(c). Pie chart showing the percentage of deletion and indels after genome editing using full USH2A exon 13 strategy.
(d). Schematic diagram of splicing acceptor targeting deletion strategy targeting the splicing site shows expected in-frame transcript with the skipped exon 13.
(e). Gel image showing fragments deletion using splicing acceptor targeting strategy in WERI-RB1 cells. Asterisks indicate fragments after deletion.
(f). Pie chart showing the percentage of deletion and indels after genome editing using paired gRNAs sgRNA-L1(sgL1), sgRNA-K4 (sgK4).
(g). Pie chart showing the percentage of deletion and indels after genome editing using paired gRNAs sgRNA-L1(sgL1), sgRNA-K5 (sgK5).
(h). NGS of edited genome DNA showing USH2A exon 13 splicing acceptor deletion after genome editing using sgRNA-L1, sgRNA-K5.

FIGS. 4A-I. Splicing acceptor targeting strategy caused small deletion results in robust generation of USH2A exon 13 skipping transcripts in human cells.
(a). Schematic diagram of exon 13-unskipped (upper) and -skipped (lower) transcripts of human USH2A.
(b). Gel image showing exon skipping efficiency with or without genome editing. Red arrows indicate USH2A transcripts.
(c). Representative Sanger sequencing data showing exon 13 skipping in USH2A transcript after genome editing.
(d). Pie chart showing the percentage of different USH2A transcripts in unedited cells.
(e). Pie chart showing the percentage of different USH2A transcripts in cells edited with paired gRNAs sgL1 and sgR2.
(f). Pie chart showing the percentage of different USH2A transcripts in cells edited with paired gRNAs sgL1 and sgK4.
(g). Pie chart showing the percentage of different USH2A transcripts in cells edited with paired gRNAs sgL1 and sgK5. This combination generates the highest percentage (73%) of exon 13-skipped USH2A transcripts in human cells.
(h). NGS data generated from cDNA derived from human cells edited with paired gRNAs sgL1 and sgK4, showing different USH2A transcripts generated by splicing acceptor targeting genome editing.
(i). NGS data generated from cDNA derived from human cells edited with paired gRNAs sgL1 and sgK5, showing different USH2A transcripts generated by splicing acceptor targeting genome editing.

FIGS. 5A-F. Splicing acceptor targeting strategy induces USH2A exon 13-skipped transcripts in human induced pluripotent stem cells (hiPSCs) derived from an USH2 patient harboring a homozygous mutation c.2299delG).
(a). Sequence information of the USH2A mutation in the hiPSCs derived from an USH2 patient.
(b). Gel image showing genome editing efficiency after editing of the hiPSCs by different dosage of CRISPR/Cas9-sgL1/sgK5 targeting the splicing acceptor site. Arrow indicates edited fragment after deletion.
(c). Pie chart showing the percentage of deletion and indels after genome editing using CRISPR/Cas9-sgL1/sgK5 with an efficiency of 75% for exon 13 skipping.
(d). Schematic diagram of USH2A transcription activation using dCas9/gRNA-directed synergistic activation mediator (SAM) system.
(e). Gel image showing exon 13 skipping in edited hiPSCs derived from an USH2 patient after SAM activation.
(f). Pie charts showing the percentage of different USH2A transcripts in edited hiPSCs derived from an USH2 patient after SAM activation. 71% of USH2A transcripts indicated exon 13 skipping.

FIGS. 6A-6B. Efficient exon skipping in inner ear organoids generated from hiPSCs derived from an USH2 patient.
(a). Gel image shows a full length USH2A transcript in a healthy control and in inner ear organoids generated from hiPSCs derived from an USH2 patient. After exon skipping, exon 13 was skipped in a majority of transcripts as shown by an arrow at day 50.
(b). Bar graph showing quantitation of the ratio between USH2A exon 13-skipped transcripts and full-length USH2A transcripts, indicating over 75% exon 13 skipping mediated by the sgRNA pair sgL1/sgK5 in inner ear organoids generated from hiPSCs derived from an USH2 patient.

FIGS. 7A-7B. Exon 5 skipping in Ush2a gene causes hearing loss in mice.
(a). Plot of auditory brainstem response (ABR) threshold shifts, showing that exon 5 skipping in the normal allele leads to hearing loss in a mouse model of Usher syndrome (WT/KO), in which one Ush2a allele is normal whereas the other allele was disrupted.
(b). Plot of distortion product otoacoustic emission (DPOAE) threshold shifts, showing that exon 5 skipping in the normal allele leads to hearing loss in a mouse model of Usher syndrome (WT/KO), in which one Ush2a allele is normal whereas the other allele was disrupted.

### DETAILED DESCRIPTION

Gene therapy for USH2 by AAV is severely hampered by the size of the USH2A cDNA (15,606 nucleotides), which exceeds by a significant margin the cargo capacity of AAVs. Alternatively, skipping exon 13 in the USH2A transcript could be a potential treatment modality in which the resulting transcript encodes a slightly shortened in-framed USHERIN protein, lacking several of the Laminin EGF-like domains. The USHERIN protein translated from exon 13-skipped transcripts retains functionality, so the exon 13 skipping strategy has therapeutic potential for any hearing loss caused by mutations of USH2A exon 13.

### Robust Exon Skipping Induction by Exon 13 Splicing Acceptor Deletion Using CRISPR/Dual-sgRNA.

Deletion of the splicing acceptor can mediate exon 13 skipping in USH2A transcripts. The splicing acceptor can be deleted by gene editing at a higher efficiency relative to deleting the entire exon 13. Cas9 and dual sgRNAs can be delivered into patients' inner ear by, e.g., AAV vector or lipid nanoparticles.

Compared to other potential treatments for Usher syndrome such as antisense oligonucleotide (AON)-based treatments, the efficacy of which is difficult to evaluate and may require multiple treatments throughout the life of the subject, the compositions and methods disclosed here provide a one-time therapeutic, including the exon 13 skipping strategy, that could have durability, even for a patient's entire lifetime. In addition, the present disclosure provides evidence of robust induction of specific target exon skipping, which can be developed into gene-editing therapies for diseases involving other splicing mutations.

Specific combinations of gRNAs mediate efficient removal of USH2A exon 13 in multiple human cell lines, e.g., WERI-RB1 cells, and human induced pluripotent stems cells (hiPSCs) derived from an Usher syndrome patient. Exon 13 removal leads to efficient production of USH2A in-frame transcripts excluding exon 13, which are shown in a mouse model to be functional and promote rescue of hearing and vision. We also generated human inner ear organoids from hiPSCs derived from an Usher syndrome patient and a healthy control individual.

The USHERIN protein encoded by USH2A (GenBank Acc No. NC_000001.11, Reference GRCh38.p7 Primary Assembly, Range 215622894-216423396, complement; SEQ ID NO:1) is a transmembrance protein anchored in the photoreceptor plasma membrane (van Wijk, E., et al., Am J Hum Genet, 2004. 74 (4): p. 738-44; Grati, M., et al., J Neurosci, 2012. 32 (41): p. 14288-93). Its extracellular portion, which accounts for over 96% of the length of the protein and projects into the periciliary matrix, is thought to have an important structural and a possible signaling role for the long-term maintenance of photoreceptors (van Wijk, E., et al., Am J Hum Genet, 2004. 74 (4): p. 738-44; Grati, M., et al., J Neurosci, 2012. 32 (41): p. 14288-93). Two isoforms of USH2A have been described. Isoform b (GenBank Acc. No. NM_206933.2 (transcript) and NP_996816.2 (protein)) is most abundantly expressed in retina and is used as the canonical, standard sequence in the literature and in this application.

### Methods of Treatment

CRISPR/Cas-based exon-skipping has been successfully used for restoring the expression of functional dystrophin and dystrophic muscle function in the Duchene muscular dystrophy mouse model. The methods described herein include methods for the treatment of disorders associated with mutations in exon 13 of the USH2A gene.

In some embodiments, the disorder is Usher syndrome, e.g., type 2 Usher syndrome. Subjects with type 2 Usher syndrome (USH2) typically have moderate to severe hearing loss at birth, and vision that becomes progressively impaired starting in adolescence. In some embodiments, the disorder is autosomal recessive retinitis pigmentosa (arRP). Generally, the methods include administering a therapeutically effective amount of a genome editing system as described herein, to a subject who is in need of, or who has been determined to be in need of, such treatment. The term “genome editing system” refers to any system having RNA-guided DNA editing activity. Genome editing systems of the present disclosure include at least two components adapted from naturally occurring CRISPR systems: a gRNA and an RNA-guided nuclease. These two components form a complex that is capable of associating with a specific nucleic acid sequence in a cell and editing the DNA in or around that nucleic acid sequence, for example by making one or more of a single-strand break (an SSB or nick), a double-strand break (a DSB) and/or a base substitution. See, e.g., WO2018/026976 for a full description of exemplary genome editing systems.

As used in this context, to “treat” means to ameliorate at least one symptom of the disorder associated with mutations in exon 13 of the USH2A gene. Often, these mutations result in hearing loss and/or loss of sight; thus, a treatment comprising administration of a therapeutic gene editing system as described herein can result in a reduction in hearing impairment and/or visual impairment; a reduction in the rate of progression of hearing loss and/or vision loss; and/or a return or approach to normal hearing and/or vision. Hearing and vision can be tested using known methods, e.g., electroretinogram, optical coherence tomography, videonystagmography, and audiology testing.

The methods can be used to treat any subject (e.g., a mammalian subject, preferably a human subject) who has a mutation in exon 13 of the USH2A gene, e.g., the c.2299delG mutation or c.2276G>T mutation, e.g., in one or both alleles of USH2A. As used herein, an “allele” is one of a pair or series of genetic variants of a polymorphism (also referred to as a mutation) at a specific genomic location. As used herein, “genotype” refers to the diploid combination of alleles for a given genetic polymorphism. A homozygous subject carries two copies of the same allele and a heterozygous subject carries two different alleles. Methods for identifying subjects with such mutations are known in the art; see, e.g., Yan et al., J Hum Genet. 2009 December; 54 (12): 732-738; Leroy et al., Exp Eye Res. 2001 May; 72 (5): 503-9; or Consugar et al., Genet Med. 2015 April; 17 (4): 253-261. For example, gel electrophoresis, capillary electrophoresis, size exclusion chromatography, sequencing, and/or arrays can be used to detect the presence or absence of the allele or genotype. Amplification of nucleic acids, where desirable, can be accomplished using methods known in the art, e.g., PCR. In one example, a sample (e.g., a sample comprising genomic DNA), is obtained from a subject. The DNA in the sample is then examined to identify or detect the presence of an allele or genotype as described herein. The allele or genotype can be identified or determined by any method described herein, e.g., by Sanger sequencing or Next Generation Sequencing (NGS). Since the exon 13 is 643 bp in size, thus the genotyping of the patients with exon 13 mutations is simple and straight forward using Sanger sequencing or NGS. Other methods can include hybridization of the gene in the genomic DNA, RNA, or cDNA to a nucleic acid probe, e.g., a DNA probe (which includes cDNA and oligonucleotide probes) or an RNA probe. The nucleic acid probe can be designed to specifically or preferentially hybridize with a particular mutation (also referred to as a polymorphic variant).

Other methods of nucleic acid analysis can include direct manual sequencing (Church and Gilbert, Proc. Natl. Acad. Sci. USA 81:1991-1995 (1988); Sanger et al., Proc. Natl. Acad. Sci. USA 74:5463-5467 (1977); Beavis et al., U.S. Pat. No. 5,288,644); automated fluorescent sequencing; single-stranded conformation polymorphism assays (SSCP) (Schafer et al., Nat. Biotechnol. 15:33-39 (1995)); clamped denaturing gel electrophoresis (CDGE); two-dimensional gel electrophoresis (2DGE or TDGE); conformational sensitive gel electrophoresis (CSGE); denaturing gradient gel electrophoresis (DGGE) (Sheffield et al., Proc. Natl. Acad. Sci. USA 86:232-236 (1989)); denaturing high performance liquid chromatography (DHPLC, Underhill et al., Genome Res. 7:996-1005 (1997)); infrared matrix-assisted laser desorption/ionization (IR-MALDI) mass spectrometry (WO 99/57318); mobility shift analysis (Orita et al., Proc. Natl. Acad. Sci. USA 86:2766-2770 (1989)); restriction enzyme analysis (Flavell et al., Cell 15:25 (1978); Geever et al., Proc. Natl. Acad. Sci. USA 78:5081 (1981)); quantitative real-time PCR (Raca et al., Genet Test 8 (4): 387-94 (2004)); heteroduplex analysis; chemical mismatch cleavage (CMC) (Cotton et al., Proc. Natl. Acad. Sci. USA 85:4397-4401 (1985)); RNase protection assays (Myers et al., Science 230:1242 (1985)); use of polypeptides that recognize nucleotide mismatches, e.g., E. coli mutS protein; allele-specific PCR, and combinations of such methods. See, e.g., Gerber et al., U.S. Patent Publication No. 2004/0014095 which is incorporated herein by reference in its entirety.

In certain aspects, the present disclosure provides AAV vectors encoding CRISPR/Cas9 genome editing systems including a first gRNA and a second gRNA, wherein the target sequence of the first gRNA is any one of SEQ ID NOs: 2-244 provided in Table 1, and the target sequence of the second gRNA is any one of SEQ ID NOs: 245-431 provided in Table 2, and provides the use of such vectors to treat USH2A-associated disease. Exemplary AAV vector genomes are known in the art; see, e.g., FIG. 9 PCT/US2019/023934, which illustrates certain fixed and variable elements of these vectors: inverted terminal repeats (ITRs), one or two gRNA sequences and promoter sequences to drive their expression, a Cas9 coding sequence and another promoter to drive its expression. Each of these elements is discussed in detail herein. Although a single vector can be used to deliver a Cas9 and two gRNAs, in some embodiments a plurality of vectors are used, e.g., wherein one vector is used to deliver Cas9, and another vector or vectors is used to deliver one or more gRNAs (e.g., one vector for one gRNA, one vector for two gRNAs, or two vectors for each of two gRNAs).

### RNA-Guided Nucleases/Cas9

Various RNA-guided nucleases can be used in the present methods, e.g., as described in WO 2018/026976. This approach can use different CRISPR proteins and their corresponding gRNAs, including Streptococcus pyogenes Cas9 (SpCas9) and engineered SpCas9 variants, Staphylococcus aureus Cas9 (SaCas9), KKH variant SaCas9 (See Kleinstiver et al., Nat Biotechnol. 2015 December; 33 (12): 1293-1298; WO 2016/141224), Cpf1 (also known as Cas12a, such as AsCpf1, LPCpf1), Cas12f (such as Un1Cas12f1, AsCas12f1) etc. In some embodiments, the RNA-guided nuclease used in the present methods and compositions is a S. aureus Cas9 or a S. pyogenes cas9. In some embodiments of this disclosure a Cas9 sequence is modified to include two nuclear localization sequences (NLSs) (e.g., PKKKRKV (SEQ ID NO: 432) at the C- and N-termini of the Cas9 protein, and a mini-polyadenylation signal (or Poly-A sequence). An exemplary NLS is SV40 large T antigen NLS (PKKKRRV (SEQ ID NO: 433)) and nucleoplasmin NLS (KRPAATKKAGQAKKKK (SEQ ID NO: 434)). Other NLSs are known in the art; see, e.g., Cokol et al., EMBO Rep. 2000 Nov. 15; 1 (5): 411-415; Freitas and Cunha, Curr Genomics. 2009 December; 10 (8): 550-557. An exemplary polyadenylation signal is TAGCAATAAAGGATCGTTTATTTTCATTGGAAGCGTGTG TTGGTTTTTTGATCAGGCGCG (SEQ ID NO: 435)). Exemplary S. aureus Cas9 sequences (both nucleotide and peptide) are described in Table 4 of WO 2018/026976, e.g., SEQ ID NOs 10 and 11 therein.

### Guide RNAs

In some embodiments, the gRNAs used in the present disclosure can be unimolecular or modular, as described below.

Described herein are approaches for treating subjects with mutations in exon 13 of USH2A that make use of dual-gRNAs for deletion of exon 13. Two gRNAs (sgRNA-L and sgRNA-R, one with target sequence in intron 12, one with target sequence in intron 13) are used in combination to delete a small DNA fragment containing the exon 13 splicing acceptor. sgRNA-L has a target sequence in intron 12 and sgRNA-R has a target sequence in exon 13. Tables 1 and 2 provide exemplary sequences for target sequences of the sgRNAs targeting intron 12 (sgRNA-L) and exon 13 (sgRNA-R), respectively. The sgRNA targets fall in the 3′ 1000 bp of intron 12 (SEQ ID NOs: 2-244) and exon 13 (SEQ ID NOs: 245-431). Note that in the sequences provided here, the actual sgRNA would have U in place of T.

In some embodiments of these methods, any combination of a first gRNA having a target sequence of any one of SEQ ID NOs: 2-244, and a second gRNA having a target sequence of any one of SEQ ID NOs: 245-431 can be used to delete a certain DNA fragment from the genome, though certain combinations may be more preferred, as exemplified below.

In some embodiments, the compositions and methods disclosed herein use Staphylococcus aureus Cas9 (SaCas9) and corresponding gRNAs. SaCas9 is one of several smaller Cas9 orthologues that are suited for viral delivery (Horvath et al., J Bacteriol 190, 1401-1412 (2008); Ran et al., Nature 520, 186-191 (2015); Zhang et al., Mol Cell 50, 488-503 (2013)). The wild type recognizes a longer NNGRRT PAM that is expected to occur once in every 32 bps of random DNA; or the alternative NNGRRA PAM. Tables 1 and 2 provide exemplary sequences for the target site in exons 12 and 13, respectively. Note that the “target site” sequences provided herein are the sequences of the gRNA (although gRNA would have U in place of T).

TABLE 1
Target sequences of exemplary sgRNA-L targeting USH2A
intron 12, Reference genome: Human GRCh38 (NCBI RefSeq NC_000001.11)
chr1: 216247225-216248226.

| | | | SEQ ID |
|---|---|---|---|
| sgRNA NO. | targetSeq | CRISPR protein | NO: |
| In12-1 | AGAGTAAGATTGGCCCCCTA | SpCas9 | 2. |
| In12-2 | CTCACAAGCAATGCCATAGG | SpCas9 | 3. |
| In12-3 | TAATTATACCTTCGTGAAGC | SpCas9 | 4. |
| In12-4 | TCTCACAAGCAATGCCATAG | SpCas9 | 5. |
| In12-5 | CTTTTTTCCCAGCTTCACGA | SpCas9 | 6. |
| In12-6 | GTTGTTTCTGTTACATATGC | SpCas9 | 7. |
| In12-7 | AATATAAATCATGCTCTCCT | SpCas9 | 8. |
| In12-8 | TTCTCACAAGCAATGCCATA | SpCas9 | 9. |
| In12-9 | AACTTATCCAGGGCAATTTT | SpCas9 | 10. |
| In12-10 | TTTCTCACAAGCAATGCCAT | SpCas9 | 11. |
| In12-11 | AATTATACCTTCGTGAAGCT | SpCas9 | 12. |
| In12-12 | GATTAAACCAAAAATTGCCC | SpCas9 | 13. |
| In12-13 | TGTGATTGCTTTATGAGCCA | SpCas9 | 14. |
| In12-14 | TTGAATTTTGAGAGTAAGAT | SpCas9 | 15 |
| In12-15 | AATTTACTTAGTGTTTAAAG | SpCas9 | 16. |
| In12-16 | TATATTTAAAAGGTGAGGAT | SpCas9 | 17. |
| In12-17 | AAAATAGAGGAGCATACAAA | SpCas9 | 18. |
| In12-18 | TCATGTACTTATCATGTTTT | SpCas9 | 19. |
| In12-19 | CTCTAAAGATGTTTAATAAA | SpCas9 | 20. |
| In12-20 | ATATATTTAAAAGGTGAGGA | SpCas9 | 21. |
| In12-21 | ATATAAATAAAACTTATCCA | SpCas9 | 22. |
| In12-22 | TTTATATTACTTCTATTTAA | SpCas9 | 23. |
| In12-23 | ATATTAATTACTTAAATGTG | SpCas9 | 24. |
| In12-24 | ATGTACTTATCATGTTTTTG | SpCas9 | 25. |
| In12-25 | TAAAATATATTTAAAAGGTG | SpCas9 | 26. |
| In12-26 | AATTGAAAATGATAAAATAG | SpCas9 | 27. |
| In12-27 | AAAAACATGATAAGTACATG | SpCas9 | 28. |
| In12-28 | TGATGGATACATTAATTAGC | SpCas9 | 29. |
| In12-29 | AATATAAATAAAACTTATCC | SpCas9 | 30. |
| In12-30 | CATGTACTTATCATGTTTTT | SpCas9 | 31. |
| In12-31 | TGGAGCTCTATTGTACAGCA | SpCas9 | 32. |
| In12-32 | ATGATAAGTACATGAGGTGA | SpCas9 | 33. |
| In12-33 | TAGACAGAATGAATAAGTTC | SpCas9 | 34. |
| In12-34 | ATAAAAATTGTATATATTTA | SpCas9 | 35. |
| In12-35 | AAAGATAAAATATATTTAAA | SpCas9 | 36. |
| In12-36 | TTTAAATATATTTTATCTTT | SpCas9 | 37. |
| In12-37 | TTAAATATATTTTATCTTTA | SpCas9 | 38. |
| In12-38 | ATAGGGGGCCAATCTTACTCT | KKH-SaCas9 | 39. |
| In12-39 | ATTCAAGATAGACGAGACACA | KKH-SaCas9 | 40. |
| In12-40 | TAAAACTTATCCAGGGCAATT | KKH-SaCas9 | 41. |
| In12-41 | AAATTTATGAAGTTCATCGCA | KKH-SaCas9 | 42. |
| In12-42 | TTTTTCCCAGCTTCACGAAGG | KKH-SaCas9 | 43. |
| In12-43 | CCCAGCTTCACGAAGGTATAA | KKH-SaCas9 | 44. |
| In12-44 | TCACAAGCAATGCCATAGGGG | KKH-SaCas9 | 45. |
| In12-45 | CTACTGCAGATGATACGAACA | KKH-SaCas9 | 46. |
| In12-46 | CGAGACACAAACAATGCTACT | KKH-SaCas9 | 47. |
| In12-47 | CTTATCCAGGGCAATTTTTGG | KKH-SaCas9 | 48. |
| In12-48 | ATATTTTGTGTTCGTATCATC | KKH-SaCas9 | 49. |
| In12-49 | ATTGTTTGTGTCTCGTCTATC | KKH-SaCas9/SaCas9 | 50. |
| In12-50 | AGCATACAAAAGGATTAAACC | KKH-SaCas9 | 51. |
| In12-51 | TTGTGTCTCGTCTATCTTGAA | KKH-SaCas9 | 52. |
| In12-52 | GGGCCAATCTTACTCTCAAAA | KKH-SaCas9 | 53. |
| In12-53 | GACACAAACAATGCTACTGCA | KKH-SaCas9 | 54. |
| In12-54 | TCTGAGTCACACAAGATGACA | KKH-SaCas9 | 55. |
| In12-55 | CTGAATCCACACATTTAAGTA | KKH-SaCas9 | 56. |
| In12-56 | TGCTTTATGAGCCAAGGAGAG | KKH-SaCas9 | 57. |
| In12-57 | GTCACACAAGATGACAAGCAA | KKH-SaCas9 | 58. |
| In12-58 | ACATACCTCTTTAAACACTAA | KKH-SaCas9 | 59. |
| In12-59 | TTAAACCAAAAATTGCCCTGG | KKH-SaCas9 | 60. |
| In12-60 | ATTACTTAAATGTGTGGATTC | KKH-SaCas9 | 61. |
| In12-61 | TTGCGATGAACTTCATAAATT | KKH-SaCas9 | 62. |
| In12-62 | CATGCTCTCCTTGGCTCATAA | KKH-SaCas9 | 63. |
| In12-63 | CTTATTTCTGAATCCACACAT | KKH-SaCas9 | 64. |
| In12-64 | GGCATTGCTTGTGAGAAAACA | KKH-SaCas9 | 65. |
| In12-65 | CAGAACATACCTCTTTAAACA | KKH-SaCas9 | 66. |
| In12-66 | TCATTTTCCCATCCTCACCTT | KKH-SaCas9 | 67. |
| In12-67 | AGAGGTATGTTCTGAGTCACA | KKH-SaCas9 | 68. |
| In12-68 | AGGATTAAACCAAAAATTGCC | KKH-SaCas9/SaCas9 | 69. |
| In12-69 | AATGACAATATTTAATTTAGC | KKH-SaCas9 | 70. |
| In12-70 | AAAATGTATATGTGTACTCCT | KKH-SaCas9 | 71. |
| In12-71 | AATAAAAGGTTAAGCTGAGTA | KKH-SaCas9 | 72. |
| In12-72 | GCAAACAGTTGTATATTAAAG | KKH-SaCas9 | 73. |
| In12-73 | TAGTGTTTAAAGAGGTATGTT | KKH-SaCas9/SaCas9 | 74. |
| In12-74 | CTTAAATGTGTGGATTCAGAA | KKH-SaCas9 | 75. |
| In12-75 | TTCACGAAGGTATAATTAAAT | KKH-SaCas9 | 76. |
| In12-76 | AATATTGAGTGTTTTCTCACA | KKH-SaCas9 | 77. |
| In12-77 | ATGTGTACTCCTTTAAATAGA | KKH-SaCas9 | 78. |
| In12-78 | AAGTGTATATGCTGTTTTCAC | KKH-SaCas9 | 79. |
| In12-79 | ATTTTAACAAATGTGCTCATT | KKH-SaCas9 | 80. |
| In12-80 | TAGCTTTAATATACAACTGTT | KKH-SaCas9 | 81. |
| In12-81 | TCTTTGCATTAAGTAATAATT | KKH-SaCas9 | 82. |
| In12-82 | TATATGTGTACTCCTTTAAAT | KKH-SaCas9 | 83. |
| In12-83 | ATATAAATAAAACTTATCCAG | KKH-SaCas9 | 84. |
| In12-84 | AAACAGCATATACACTTATTT | KKH-SaCas9/SaCas9 | 85. |
| In12-85 | GATAAGAAATCTCTAAAGATG | KKH-SaCas9 | 86. |
| In12-86 | TTCTAAAGATAAGAAATCTCT | KKH-SaCas9 | 87. |
| In12-87 | CAGTTGTATATTAAAGCTAAA | KKH-SaCas9 | 88. |
| In12-88 | AGATGATACGAACACAAAATA | KKH-SaCas9/SaCas9 | 89. |
| In12-89 | GGATGGGAAAATGATTTCATT | KKH-SaCas9 | 90. |
| In12-90 | ATTTCTGAATCCACACATTTA | KKH-SaCas9 | 91. |
| In12-91 | GATGTTTAATAAAAGGTTAAG | KKH-SaCas9/SaCas9 | 92. |
| In12-92 | ATCTTACTCTCAAAATTCAAT | KKH-SaCas9 | 93. |
| In12-93 | TTTAAAAGGTGAGGATGGGAA | KKH-SaCas9 | 94. |
| In12-94 | CAGAATTTACTTAGTGTTTAA | KKH-SaCas9 | 95. |
| In12-95 | ATGCTCCTCTATTTTATCATT | KKH-SaCas9 | 96. |
| In12-96 | CGTGAAGCTGGGAAAAAAGAA | KKH-SaCas9 | 97. |
| In12-97 | AATCTCTAAAGATGTTTAATA | KKH-SaCas9 | 98. |
| In12-98 | TAGTAGAATTACATATAACAA | KKH-SaCas9 | 99. |
| In12-99 | ATGCAAAGAAAAATGCTTAAT | KKH-SaCas9 | 100. |
| In12-100 | AGAGCATGATTTATATTAATT | KKH-SaCas9 | 101. |
| In12-101 | ATTTGTTATATGTAATTCTAC | KKH-SaCas9 | 102. |
| In12-102 | CTGGAGCTCTTTTTCTCTTTA | KKH-SaCas9 | 103. |
| In12-103 | GCATTTTTCTTTGCATTAAGT | KKH-SaCas9 | 104 |
| In12-104 | TCTCAAAATTCAATGACAATA | KKH-SaCas9 | 105. |
| In12-105 | ATATTTAAAAGGTGAGGATGG | KKH-SaCas9 | 106. |
| In12-106 | TGTATATGCTGTTTTCACAAA | KKH-SaCas9 | 107. |
| In12-107 | CCCAAAAACATGATAAGTACA | KKH-SaCas9 | 108. |
| In12-108 | TGTGCTCATTTAAAATTATAG | KKH-SaCas9/SaCas9 | 109 |
| In12-109 | TAATTATTACTTAATGCAAAG | KKH-SaCas9 | 110. |
| In12-110 | GCATGATTTATATTAATTGAA | KKH-SaCas9 | 111. |
| In12-111 | TTTCTTTTTTCCCAGCTTCAC | KKH-SaCas9 | 112. |
| In12-112 | CATTAAGCATTTTTCTTTGCA | KKH-SaCas9 | 113. |
| In12-113 | CAACTGTTTGCGATGAACTTC | KKH-SaCas9 | 114. |
| In12-114 | ATTATAAAATGATTAATTCCA | KKH-SaCas9 | 115. |
| In12-115 | AAAAACAACTAATTTGTTATA | KKH-SaCas9 | 116. |
| In12-116 | ATTTTAAATGAGCACATTTGT | KKH-SaCas9 | 117. |
| In12-117 | TTAAATGAGCACATTTGTTAA | KKH-SaCas9 | 118. |
| In12-118 | AAGCTAAATTAAATATTGTCA | KKH-SaCas9/SaCas9 | 119. |
| In12-119 | TCCTCTATTTTATCATTTTCA | KKH-SaCas9 | 120. |
| In12-120 | ATATGTAATTCTACTATAATT | KKH-SaCas9 | 121. |
| In12-121 | ATTGTCATTGAATTTTGAGAG | KKH-SaCas9 | 122. |
| In12-122 | CCACACATTTAAGTAATTAAT | KKH-SaCas9 | 123. |
| In12-123 | TTGGGGTGAGAACATTTAAGA | KKH-SaCas9 | 124. |
| In12-124 | AACCTTTTATTAAACATCTTT | KKH-SaCas9 | 125. |
| In12-125 | ATTATAGTAGAATTACATATA | KKH-SaCas9 | 126. |
| In12-126 | TAAGCATTTTTCTTTGCATTA | KKH-SaCas9 | 127. |
| In12-127 | TTAGTTGTTTTTCTAAAGATA | KKH-SaCas9 | 128. |
| In12-128 | TTATATTAATTACTTAAATGT | KKH-SaCas9/SaCas9 | 129. |
| In12-129 | GTATATATTTATGGCAGACAA | KKH-SaCas9 | 130. |
| In12-130 | AAAAACATGATAAGTACATGA | KKH-SaCas9 | 131. |
| In12-131 | TGTTCTCACCCCAAAAACATG | KKH-SaCas9 | 132. |
| In12-132 | GAGTACAAAGTTTCAGCTAGA | KKH-SaCas9/SaCas9 | 133. |
| In12-133 | TAACAAATTAGTTGTTTTTCT | KKH-SaCas9 | 134. |
| In12-134 | ACAAATGTGCTCATTTAAAAT | KKH-SaCas9 | 135. |
| In12-135 | TACTTAATGCAAAGAAAAATG | KKH-SaCas9 | 136. |
| In12-136 | ATTTTATCATTTTCAATTAAT | KKH-SaCas9 | 137. |
| In12-137 | GTGATGGATACATTAATTAGC | KKH-SaCas9/SaCas9 | 138. |
| In12-138 | CTCATGTACTTATCATGTTTT | KKH-SaCas9/SaCas9 | 139. |
| In12-139 | TAATATAAAAAACAGAATTTA | KKH-SaCas9 | 140. |
| In12-140 | AGTTTTATTTATATTAATTAC | KKH-SaCas9 | 141. |
| In12-141 | ACATGATAAGTACATGAGGTG | KKH-SaCas9/SaCas9 | 142. |
| In12-142 | CAAATCTTAAAAACTATTTTA | KKH-SaCas9 | 143. |
| In12-143 | CACATTTGTTAAAATAGTTTT | KKH-SaCas9 | 144. |
| In12-144 | TAAATATTGTCATTGAATTTT | KKH-SaCas9/SaCas9 | 145. |
| In12-145 | ATTTATATTAATTGAAAATGA | KKH-SaCas9 | 146. |
| In12-146 | ATAAAATAGAGGAGCATACAA | KKH-SaCas9/SaCas9 | 147. |
| In12-147 | CAAAACATCATGTTGTCTGCC | KKH-SaCas9 | 148. |
| In12-148 | TGATATATGTACACATTATAA | KKH-SaCas9 | 149. |
| In12-149 | ACATTTAAGATCTAATCTCTT | KKH-SaCas9 | 150. |
| In12-150 | AGTTTCAGCTAGACAGAATGA | KKH-SaCas9 | 151. |
| In12-151 | GATAAAATATATTTAAAAGGT | KKH-SaCas9/SaCas9 | 152. |
| In12-152 | CCTAAAGATAAAATATATTTA | KKH-SaCas9 | 153. |
| In12-153 | TTTTATATTACTTCTATTTAA | KKH-SaCas9/SaCas9 | 154. |
| In12-154 | CATAAATATATACAATTTTTA | KKH-SaCas9 | 155. |
| In12-155 | ATGTTGTCTGCCATAAATATA | KKH-SaCas9 | 156. |
| In12-156 | TAAATGTTCTCACCCCAAAAA | KKH-SaCas9 | 157. |
| In12-157 | AAATTGCTAAGAGATTAGATC | KKH-SaCas9 | 158. |
| In12-158 | CAAGTATACAATACATTATTA | KKH-SaCas9 | 159. |
| In12-159 | ACAAAGTTTCAGCTAGACAGA | KKH-SaCas9/SaCas9 | 160. |
| In12-160 | TTCATAAATTTTTAATTATTA | KKH-SaCas9 | 161. |
| In12-161 | ATTAGCTGGAATTAATCATTT | KKH-SaCas9 | 162. |
| In12-162 | AGTACATGAGGTGATGGATAC | KKH-SaCas9 | 163. |
| In12-163 | TTGTATACTTGAAAATTGCTA | KKH-SaCas9 | 164. |
| In12-164 | AGCATGGTGACTATACTTAAT | KKH-SaCas9 | 165. |
| In12-165 | TACAGCATGGTGACTATACTT | KKH-SaCas9 | 166. |
| In12-166 | AAATAGAAGTAATATAAAAAA | KKH-SaCas9/SaCas9 | 167. |
| In12-167 | GGAAAAAAGAAAAAAATGTCA | KKH-SaCas9 | 168. |
| In12-168 | TTATGGCAGACAACATGATGT | KKH-SaCas9 | 169. |
| In12-169 | TTTTGATATATGTACACATTA | KKH-SaCas9 | 170. |
| In12-170 | TGGATACATTAATTAGCTGGA | KKH-SaCas9 | 171. |
| In12-171 | ATACAATACATTATTATTAAG | KKH-SaCas9 | 172. |
| In12-172 | TTCTGGAGCTCTATTGTACAG | KKH-SaCas9 | 173. |
| In12-173 | TAAGTAATAATTAAAAATTTA | KKH-SaCas9 | 174. |
| In12-174 | TGTTTTTGGGGTGAGAACATT | KKH-SaCas9 | 175. |
| In12-175 | TACTTGAAAATTGCTAAGAGA | KKH-SaCas9 | 176. |
| In12-176 | ATAATAATGTATTGTATACTT | KKH-SaCas9 | 177. |
| In12-177 | GATCTAATCTCTTAGCAATTT | KKH-SaCas9 | 178. |
| In12-178 | TCTCTTAGCAATTTTCAAGTA | KKH-SaCas9 | 179. |
| In12-179 | TTGTACAGCATGGTGACTATA | KKH-SaCas9 | 180. |
| In12-180 | TTAAGTATAGTCACCATGCTG | KKH-SaCas9 | 181. |
| In12-181 | AATAGAAGTAATATAAAAAACAG | Cpf1/Cas12f | 182. |
| In12-182 | AAGGAGTACACATATACATTTTA | Cpf1/Cas12f | 183. |
| In12-183 | TATTACTTCTATTTAAAGGAGTA | Cpf1/Cas12f | 184. |
| In12-184 | CTTAGTGTTTAAAGAGGTATGTT | Cpf1/Cas12f | 185. |
| In12-185 | AAGAGGTATGTTCTGAGTCACAC | Cpf1/Cas12f | 186. |
| In12-186 | AACACTAAGTAAATTCTGTTTTT | Cpf1/Cas12f | 187. |
| In12-187 | TGAGCCAAGGAGAGCATGATTTA | Cpf1/Cas12f | 188. |
| In12-188 | TATTAATTGAAAATGATAAAATA | Cpf1/Cas12f | 189. |
| In12-189 | AATTAATATAAATCATGCTCTCC | Cpf1/Cas12f | 190. |
| In12-190 | TCATTTTCAATTAATATAAATCA | Cpf1/Cas12f | 191. |
| In12-191 | TATGCTCCTCTATTTTATCATTT | Cpf1/Cas12f | 192. |
| In12-192 | ATCCTTTTGTATGCTCCTCTATT | Cpf1/Cas12f | 193. |
| In12-193 | GTTTAATCCTTTTGTATGCTCCT | Cpf1/Cas12f | 194. |
| In12-194 | TTTATATTAATTACTTAAATGTG | Cpf1/Cas12f | 195. |
| In12-195 | TATTAATTACTTAAATGTGTGGA | Cpf1/Cas12f | 196. |
| In12-196 | AGTAATTAATATAAATAAAACTT | Cpf1/Cas12f | 197. |
| In12-197 | TGAATCCACACATTTAAGTAATT | Cpf1/Cas12f | 198. |
| In12-198 | ACAAAAATAGTTATCAGCTGACA | Cpf1/Cas12f | 199. |
| In12-199 | TGAAAACAGCATATACACTTATT | Cpf1/Cas12f | 200. |
| In12-200 | TTTTTTCCCAGCTTCACGAAGGT | Cpf1/Cas12f | 201. |
| In12-201 | CCAGCTTCACGAAGGTATAATTA | Cpf1/Cas12f | 202. |
| In12-202 | ATTATACCTTCGTGAAGCTGGGA | Cpf1/Cas12f | 203. |
| In12-203 | TTTAATTATACCTTCGTGAAGCT | Cpf1/Cas12f | 204. |
| In12-204 | TGGCAGACAACATGATGTTTTGT | Cpf1/Cas12f | 205. |
| In12-205 | ATATATGTACACATTATAAAATG | Cpf1/Cas12f | 206. |
| In12-206 | TAATGTGTACATATATCAAAACA | Cpf1/Cas12f | 207. |
| In12-207 | GGGTGAGAACATTTAAGATCTAA | Cpf1/Cas12f | 208. |
| In12-208 | AGATCTAATCTCTTAGCAATTTT | Cpf1/Cas12f | 209. |
| In12-209 | AAGTATACAATACATTATTATTA | Cpf1/Cas12f | 210. |
| In12-210 | AGCTAGACAGAATGAATAAGTTC | Cpf1/Cas12f | 211. |
| In12-211 | TACTCAGCTTAACCTTTTATTAA | Cpf1/Cas12f | 212. |
| In12-212 | TTAAACATCTTTAGAGATTTCTT | Cpf1/Cas12f | 213. |
| In12-213 | ATAAAAGGTTAAGCTGAGTACAA | Cpf1/Cas12f | 214. |
| In12-214 | GAGATTTCTTATCTTTAGAAAAA | Cpf1/Cas12f | 215. |
| In12-215 | TTATCTTTAGAAAAACAACTAAT | Cpf1/Cas12f | 216. |
| In12-216 | GAAAAACAACTAATTTGTTATAT | Cpf1/Cas12f | 217. |
| In12-217 | TAAAGATAAGAAATCTCTAAAGA | Cpf1/Cas12f | 218. |
| In12-218 | TTATATGTAATTCTACTATAATT | Cpf1/Cas12f | 219. |
| In12-219 | AATGAGCACATTTGTTAAAATAG | Cpf1/Cas12f | 220. |
| In12-220 | AAATTATAGTAGAATTACATATA | Cpf1/Cas12f | 221. |
| In12-221 | TTAAAATAGTTTTTAAGATTTGT | Cpf1/Cas12f | 222. |
| In12-222 | ACAAATGTGCTCATTTAAAATTA | Cpf1/Cas12f | 223. |
| In12-223 | AGATTTGTTAAAGAGAAAAAGAG | Cpf1/Cas12f | 224. |
| In12-224 | TTAAAGAGAAAAAGAGCTCCAGC | Cpf1/Cas12f | 225. |
| In12-225 | ACAAATCTTAAAAACTATTTTAA | Cpf1/Cas12f | 226. |
| In12-226 | TCTTTAACAAATCTTAAAAACTA | Cpf1/Cas12f | 227. |
| In12-227 | TGTTACATATGCTGGAGCTCTTT | Cpf1/Cas12f | 228. |
| In12-228 | CATTAAGCATTTTTCTTTGCATT | Cpf1/Cas12f | 229. |
| In12-229 | TTTGCATTAAGTAATAATTAAAA | Cpf1/Cas12f | 230. |
| In12-230 | CATTAAGTAATAATTAAAAATTT | Cpf1/Cas12f | 231. |
| In12-231 | ATTATTACTTAATGCAAAGAAAA | Cpf1/Cas12f | 232. |
| In12-232 | TGAAGTTCATCGCAAACAGTTGT | Cpf1/Cas12f | 233. |
| In12-233 | CGATGAACTTCATAAATTTTTAA | Cpf1/Cas12f | 234. |
| In12-234 | ATATACAACTGTTTGCGATGAAC | Cpf1/Cas12f | 235. |
| In12-235 | GCTTTAATATACAACTGTTTGCG | Cpf1/Cas12f | 236. |
| In12-236 | ATTTAGCTTTAATATACAACTGT | Cpf1/Cas12f | 237. |
| In12-237 | AGAGTAAGATTGGCCCCCTATGG | Cpf1/Cas12f | 238. |
| In12-238 | TCACAAGCAATGCCATAGGGGGC | Cpf1/Cas12f | 239. |
| In12-239 | TGTTCGTATCATCTGCAGTAGCA | Cpf1/Cas12f | 240. |
| In12-240 | TGTCTCGTCTATCTTGAATGAAA | Cpf1/Cas12f | 241. |
| In12-241 | ATTCAAGATAGACGAGACACAAA | Cpf1/Cas12f | 242. |
| In12-242 | CCATCCTCACCTTTTAAATATAT | Cpf1/Cas12f | 243. |
| In12-243 | AAAGGTGAGGATGGGAAAATGAT | Cpf1/Cas12f | 244. |

TABLE 2
Target sequences of exemplary sgRNA-R targeting USH2A
exon 13, Reference genome: Human GRCh38 (NCBI RefSeq NC_000001.11)
chr1: 216246565-216247246.

| | | | SEQ ID |
|---|---|---|---|
| sgRNA NO. | targetSeq | CRISPR protein | NO: |
| Ex13-1 | CTGTGTGTGCCTAATCGTCA | SpCas9 | 245. |
| Ex13-2 | CCTTGCAAATTAGGGGTAAC | SpCas9 | 246. |
| Ex13-3 | CTTGACGATTAGGCACACAC | SpCas9 | 247. |
| Ex13-4 | CTCCGAAGCTTTAATGATGT | SpCas9 | 248. |
| Ex13-5 | CCTGTTACCCCTAATTTGCA | SpCas9 | 249. |
| Ex13-6 | CACCTTCTTCCTTGACGATT | SpCas9 | 250. |
| Ex13-7 | GATTCCTTGGGGACATTACC | SpCas9 | 251. |
| Ex13-8 | AGGTGTAATCAGTGTCAACC | SpCas9 | 252. |
| Ex13-9 | ACTGTCTGTAATGCTAAGAC | SpCas9 | 253. |
| Ex13-10 | CCAGTCTTATCACAGTTGCA | SpCas9 | 254. |
| Ex13-11 | ACAGTCACAGGCCTTACAAT | SpCas9 | 255. |
| Ex13-12 | GGAAAGAATTATTTTGCCGT | SpCas9 | 256. |
| Ex13-13 | CCTTGCAACTGTGATAAGAC | SpCas9 | 257. |
| Ex13-14 | GTTAGATGTCACCAATTGTA | SpCas9 | 258. |
| Ex13-15 | ATGGTCAAATTGTACCTGTG | SpCas9 | 259. |
| Ex13-16 | CCATGGAGGTTACACTGGCA | SpCas9 | 260. |
| Ex13-17 | GCCATGGAGGTTACACTGGC | SpCas9 | 261. |
| Ex13-18 | TAGCATTACAGACAGTCCCA | SpCas9 | 262. |
| Ex13-19 | ATTGGGTCACAAATGGTCCC | SpCas9 | 263. |
| Ex13-20 | ATTCCTTGGGGACATTACCT | SpCas9 | 264. |
| Ex13-21 | CTGTCTGTAATGCTAAGACA | SpCas9 | 265. |
| Ex13-22 | CTTGCAACTGTGATAAGACT | SpCas9 | 266. |
| Ex13-23 | ACAGGCACTGGCCACTGATT | SpCas9 | 267. |
| Ex13-24 | ACCATTTGTGACCCAATCAG | SpCas9 | 268. |
| Ex13-25 | TGCCTAATCGTCAAGGAAGA | SpCas9 | 269. |
| Ex13-26 | GGTGTCACACTGAAGTCCTT | SpCas9 | 270. |
| Ex13-27 | ATCTGCAAGCCCAATGTTGA | SpCas9 | 271. |
| Ex13-28 | GCCACTGATTGGGTCACAAA | SpCas9 | 272. |
| Ex13-29 | CACTGTCTCCCTTCAACATT | SpCas9 | 273. |
| Ex13-30 | TGGAGGGAAACTTCTACCTA | SpCas9 | 274. |
| Ex13-31 | CTGCTGTGTAACAAATCAAC | SpCas9 | 275. |
| Ex13-32 | ACAATGTCCTTGCAAATTAG | SpCas9 | 276. |
| Ex13-33 | GTAATCAGTGTGAGCCTCAC | SpCas9 | 277. |
| Ex13-34 | CTGAGCCATGGAGGTTACAC | SpCas9 | 278. |
| Ex13-35 | GCACTGTCTCCCTTCAACAT | SpCas9 | 279. |
| Ex13-36 | GACAATGTCCTTGCAAATTA | SpCas9 | 280. |
| Ex13-37 | AAATTCTGCAATCCTCACTC | SpCas9 | 281. |
| Ex13-38 | GGACAATGTCCTTGCAAATT | SpCas9 | 282. |
| Ex13-39 | CACAGGCACTGGCCACTGAT | SpCas9 | 283. |
| Ex13-40 | AGGAATCACACTCACACATC | SpCas9 | 284. |
| Ex13-41 | GGTCCCAGGTAATGTCCCCA | SpCas9 | 285. |
| Ex13-42 | CCCTGCCAGTGTAACCTCCA | SpCas9 | 286. |
| Ex13-43 | GATAAGACTGGGACAATAAA | SpCas9 | 287. |
| Ex13-44 | AGGTGTGATCATTGCAATTT | SpCas9 | 288. |
| Ex13-45 | GATGTGTGAGTGTGATTCCT | SpCas9 | 289. |
| Ex13-46 | ATTTGTTCACTGAGCCATGG | SpCas9 | 290. |
| Ex13-47 | GACACAGCTGGATCCCTCCC | SpCas9 | 291. |
| Ex13-48 | CAGTGCAATAAATGTTTGGA | SpCas9 | 292. |
| Ex13-49 | ATCCAACATCATTAAAGCTT | SpCas9 | 293. |
| Ex13-50 | AGAATTTGTTCACTGAGCCA | SpCas9 | 294. |
| Ex13-51 | CCCATAAAAGTTTTCTCTGC | SpCas9 | 295. |
| Ex13-52 | AATTCTGCAATCCTCACTCT | SpCas9 | 296. |
| Ex13-53 | CCTGCAGAGAAAACTTTTAT | SpCas9 | 297. |
| Ex13-54 | CATTACAGACAGTCCCAGGG | SpCas9 | 298. |
| Ex13-55 | GAGACAGTGCAATAAATGTT | SpCas9 | 299. |
| Ex13-56 | TTAGCATTACAGACAGTCCC | SpCas9 | 300. |
| Ex13-57 | ACAGTGCAATAAATGTTTGG | SpCas9 | 301. |
| Ex13-58 | CACTCACACTGCCCAGAGTG | SpCas9 | 302. |
| Ex13-59 | TCTGCAAGCCCAATGTTGAA | SpCas9 | 303. |
| Ex13-60 | TAATACATTTCTTTCTTACC | SpCas9 | 304. |
| Ex13-61 | ATGTGTGAGTGTGATTCCTT | SpCas9 | 305. |
| Ex13-62 | TGTGTGAGTGTGATTCCTTG | SpCas9 | 306. |
| Ex13-63 | ATTACAGACAGTCCCAGGGA | SpCas9 | 307. |
| Ex13-64 | TCCAGCTGTGTCACAGTCAC | SpCas9 | 308. |
| Ex13-65 | ACACAGCTGGATCCCTCCCT | SpCas9 | 309. |
| Ex13-66 | ACCTGCAGAGAAAACTTTTA | SpCas9 | 310. |
| Ex13-67 | GCCTGTGACTGTGACACAGC | SpCas9 | 311. |
| Ex13-68 | GATTAGGCACACACAGGCAC | SpCas9 | 312. |
| Ex13-69 | ATATTTTATCTTTAGGGCTT | SpCas9 | 313. |
| Ex13-70 | CACAGTTGCAAGGCAGACAG | SpCas9 | 314. |
| Ex13-71 | GAGTGCAAAAAAGAAGCCAA | SpCas9 | 315. |
| Ex13-72 | GCAGTGTTGAAAATTGTCAA | SpCas9 | 316. |
| Ex13-73 | TTAGGGGTAACAGGTCTTCGC | KKH-SaCas9 | 317. |
| Ex13-74 | GCACTGGCCACTGATTGGGTC | KKH-SaCas9 | 318. |
| Ex13-75 | CCAACATCATTAAAGCTTCGG | KKH-SaCas9 | 319. |
| Ex13-76 | GAGGGAAACTTCTACCTACGG | KKH-SaCas9 | 320. |
| Ex13-77 | TTACAGCGAAGACCTGTTACC | KKH-SaCas9 | 321. |
| Ex13-78 | TGGTCCCAGGTAATGTCCCCA | KKH-SaCas9/SaCas9 | 322. |
| Ex13-79 | GGAAACTTCTACCTACGGCAA | KKH-SaCas9 | 323. |
| Ex13-80 | ACAGGGCAGTGCATCTGCAAG | KKH-SaCas9 | 324. |
| Ex13-81 | GTAACCTCCATGGCTCAGTGA | KKH-SaCas9 | 325. |
| Ex13-82 | ATTACCTGGGACCATTTGTGA | KKH-SaCas9 | 326. |
| Ex13-83 | CTGCCAGTGTAACCTCCATGG | KKH-SaCas9 | 327. |
| Ex13-84 | AATTGGTGACATCTAACCCAT | KKH-SaCas9 | 328. |
| Ex13-85 | GTCACAGTCACAGGCCTTACA | KKH-SaCas9 | 329. |
| Ex13-86 | GGATCCCTCCCTGGGACTGTC | KKH-SaCas9 | 330. |
| Ex13-87 | ATGATGTTGGATGTGAGCCCT | KKH-SaCas9 | 331. |
| Ex13-88 | CACTGCCCAGAGTGAGGATTG | KKH-SaCas9/SaCas9 | 332. |
| Ex13-89 | CTGCCCTGTCTTAGCATTACA | KKH-SaCas9 | 333. |
| Ex13-90 | TGTCCTTGCAAATTAGGGGTA | KKH-SaCas9 | 334. |
| Ex13-91 | AATTCTGCAATCCTCACTCTG | KKH-SaCas9 | 335. |
| Ex13-92 | ATCACACCTAAGCCCTAAAGA | KKH-SaCas9 | 336. |
| Ex13-93 | TGCACTCACACTGCCCAGAGT | KKH-SaCas9/SaCas9 | 337. |
| Ex13-94 | CTGTCTGTAATGCTAAGACAG | KKH-SaCas9 | 338. |
| Ex13-95 | ACTCACACATCTGGCAGTGTT | KKH-SaCas9 | 339. |
| Ex13-96 | GCAATCCTCACTCTGGGCAGT | KKH-SaCas9/SaCas9 | 340. |
| Ex13-97 | CTGTGTCACAGTCACAGGCCT | KKH-SaCas9 | 341. |
| Ex13-98 | TTCTCCGAAGCTTTAATGATG | KKH-SaCas9/SaCas9 | 342. |
| Ex13-99 | AATTGTACCTGTGAGGCTCAC | KKH-SaCas9 | 343. |
| Ex13-100 | GTGTGCCTAATCGTCAAGGAA | KKH-SaCas9 | 344. |
| Ex13-101 | CATGGCTCAGTGAACAAATTC | KKH-SaCas9 | 345. |
| Ex13-102 | GCTGTAATCAGTGTGAGCCTC | KKH-SaCas9 | 346. |
| Ex13-103 | CTGGCCACTGATTGGGTCACA | KKH-SaCas9 | 347. |
| Ex13-104 | TTTTCTCTGCAGGTGTCACAC | KKH-SaCas9 | 348. |
| Ex13-105 | ATTGGGTCACAAATGGTCCCA | KKH-SaCas9 | 349. |
| Ex13-106 | CTGATTGGGTCACAAATGGTC | KKH-SaCas9 | 350. |
| Ex13-107 | CCATTTGTGACCCAATCAGTG | KKH-SaCas9 | 351. |
| Ex13-108 | TTAGGTGTGATCATTGCAATT | KKH-SaCas9/SaCas9 | 352. |
| Ex13-109 | GGATTTAAATTTCTCCGAAGC | KKH-SaCas9 | 353. |
| Ex13-110 | AGGCCTGTGACTGTGACACAG | KKH-SaCas9/SaCas9 | 354. |
| Ex13-111 | GCAAGCCCAATGTTGAAGGGA | KKH-SaCas9 | 355. |
| Ex13-112 | CACACAGGCACTGGCCACTGA | KKH-SaCas9/SaCas9 | 356. |
| Ex13-113 | TTAGGGCTTAGGTGTGATCAT | KKH-SaCas9 | 357. |
| Ex13-114 | TTCCTCTGTCTGCCTTGCAAC | KKH-SaCas9 | 358. |
| Ex13-115 | CACAGGTACAATTTGACCATT | KKH-SaCas9 | 359. |
| Ex13-116 | AGAAGGTGTAATCAGTGTCAA | KKH-SaCas9 | 360. |
| Ex13-117 | CTTTTTTGCACTCACACTGCC | KKH-SaCas9/SaCas9 | 361. |
| Ex13-118 | GCATTACAGACAGTCCCAGGG | KKH-SaCas9/SaCas9 | 362. |
| Ex13-119 | TCTCCCTTCAACATTGGGCTT | KKH-SaCas9 | 363. |
| Ex13-120 | AGGCACACACAGGCACTGGCC | KKH-SaCas9 | 364. |
| Ex13-121 | AGAATTTGTTCACTGAGCCAT | KKH-SaCas9 | 365. |
| Ex13-122 | CCTAATCGTCAAGGAAGAAGG | KKH-SaCas9 | 366. |
| Ex13-123 | AATCAGTGTGAGCCTCACAGG | KKH-SaCas9 | 367. |
| Ex13-124 | CAATGATCACACCTAAGCCCT | KKH-SaCas9 | 368. |
| Ex13-125 | ATTTTATCTTTAGGGCTTAGG | KKH-SaCas9 | 369. |
| Ex13-126 | TAAATGGCTCTCTGCTGTGTA | KKH-SaCas9 | 370. |
| Ex13-127 | CTGGCAGTGTTGAAAATTGTC | KKH-SaCas9 | 371. |
| Ex13-128 | CATCTGGCAGTGTTGAAAATT | KKH-SaCas9 | 372. |
| Ex13-129 | CAACACTGCCAGATGTGTGAG | KKH-SaCas9 | 373. |
| Ex13-130 | AGCTTCGGAGAAATTTAAATC | KKH-SaCas9 | 374. |
| Ex13-131 | AAGAATTATTTTGCCGTAGGT | KKH-SaCas9 | 375. |
| Ex13-132 | AATTTGCAAGGACATTGTCCT | KKH-SaCas9 | 376. |
| Ex13-133 | AGGAATCACACTCACACATCT | KKH-SaCas9 | 377. |
| Ex13-134 | AGGACAATGTCCTTGCAAATT | KKH-SaCas9/SaCas9 | 378. |
| Ex13-135 | AGTGGCCAGTGCCTGTGTGTG | KKH-SaCas9 | 379. |
| Ex13-136 | TTTAAATTTCTCCGAAGCTTT | KKH-SaCas9 | 380. |
| Ex13-137 | ATGTTGAAGGGAGACAGTGCA | KKH-SaCas9 | 381. |
| Ex13-138 | TTGCAACTGTGATAAGACTGG | KKH-SaCas9 | 382. |
| Ex13-139 | AACTGTGATAAGACTGGGACA | KKH-SaCas9 | 383. |
| Ex13-140 | TGCTGTGTAACAAATCAACAG | KKH-SaCas9 | 384. |
| Ex13-141 | TCATTAAAGCTTCGGAGAAAT | KKH-SaCas9 | 385. |
| Ex13-142 | CAGCAGAGAGCCATTTATTGT | KKH-SaCas9 | 386. |
| Ex13-143 | TCAGTGTCAACCAGGTAAGAA | KKH-SaCas9 | 387. |
| Ex13-144 | CAGGGAGGGATCCAGCTGTGT | KKH-SaCas9 | 388. |
| Ex13-145 | AATCAACAGGACAATGTCCTT | KKH-SaCas9 | 389. |
| Ex13-146 | ACACCTGCAGAGAAAACTTTT | KKH-SaCas9/SaCas9 | 390. |
| Ex13-147 | CATTTATTGTCCCAGTCTTAT | KKH-SaCas9 | 391. |
| Ex13-148 | ATTTTCAACACTGCCAGATGT | KKH-SaCas9/SaCas9 | 392. |
| Ex13-149 | AGTTGCAAGGCAGACAGAGGA | KKH-SaCas9/SaCas9 | 393. |
| Ex13-150 | AGTGTTGAAAATTGTCAATGG | KKH-SaCas9 | 394. |
| Ex13-151 | CCCAATGTTGAAGGGAGACAG | KKH-SaCas9 | 395. |
| Ex13-152 | TTTCTTTCTTACCTGGTTGAC | KKH-SaCas9 | 396. |
| Ex13-153 | AATATATTTTATCTTTAGGGC | KKH-SaCas9 | 397. |
| Ex13-154 | CATTGACAATTTTCAACACTG | KKH-SaCas9 | 398. |
| Ex13-155 | TAACCCATAAAAGTTTTCTCT | KKH-SaCas9 | 399. |
| Ex13-156 | TGCAGAGAAAACTTTTATGGG | KKH-SaCas9 | 400. |
| Ex13-157 | GTGATCATTGCAATTTTGGAT | KKH-SaCas9 | 401. |
| Ex13-158 | GCAAAAAAGAAGCCAAAGGAC | KKH-SaCas9 | 402. |
| Ex13-159 | GAAATTTAAATCCAAAATTGC | KKH-SaCas9 | 403. |
| Ex13-160 | ACTGATTACACCTTCTTCCTT | KKH-SaCas9 | 404. |
| Ex13-161 | AGAGGAAAGAATTATTTTGCC | KKH-SaCas9 | 405. |
| Ex13-162 | GGAGAAATTTAAATCCAAAAT | KKH-SaCas9 | 406. |
| Ex13-163 | TCTTTAGGGCTTAGGTGTGATCA | Cpf1/Cas12f | 407. |
| Ex13-164 | GGGCTTAGGTGTGATCATTGCAA | Cpf1/Cas12f | 408. |
| Ex13-165 | GATTTAAATTTCTCCGAAGCTTT | Cpf1/Cas12f | 409. |
| Ex13-166 | AATTTCTCCGAAGCTTTAATGAT | Cpf1/Cas12f | 410. |
| Ex13-167 | AATCCAAAATTGCAATGATCACA | Cpf1/Cas12f | 411. |
| Ex13-168 | TCCGAAGCTTTAATGATGTTGGA | Cpf1/Cas12f | 412. |
| Ex13-169 | ATGATGTTGGATGTGAGCCCTGC | Cpf1/Cas12f | 413. |
| Ex13-170 | TTCACTGAGCCATGGAGGTTACA | Cpf1/Cas12f | 414. |
| Ex13-171 | CACTCACACTGCCCAGAGTGAGG | Cpf1/Cas12f | 415. |
| Ex13-172 | GCTTCTTTTTTGCACTCACACTG | Cpf1/Cas12f | 416. |
| Ex13-173 | TCTGCAGGTGTCACACTGAAGTC | Cpf1/Cas12f | 417. |
| Ex13-174 | TGGGTTAGATGTCACCAATTGTA | Cpf1/Cas12f | 418. |
| Ex13-175 | TTGCACTGTCTCCCTTCAACATT | Cpf1/Cas12f | 419. |
| Ex13-176 | GAGGGAAACTTCTACCTACGGCA | Cpf1/Cas12f | 420. |
| Ex13-177 | CCTCCAAACATTTATTGCACTGT | Cpf1/Cas12f | 421. |
| Ex13-178 | CCGTAGGTAGAAGTTTCCCTCCA | Cpf1/Cas12f | 422. |
| Ex13-179 | CTCTGTCTGCCTTGCAACTGTGA | Cpf1/Cas12f | 423. |
| Ex13-180 | TTGTCCCAGTCTTATCACAGTTG | Cpf1/Cas12f | 424. |
| Ex13-181 | TTACACAGCAGAGAGCCATTTAT | Cpf1/Cas12f | 425. |
| Ex13-182 | CAAGGACATTGTCCTGTTGATTT | Cpf1/Cas12f | 426. |
| Ex13-183 | ACCATTGACAATTTTCAACACTG | Cpf1/Cas12f | 427. |
| Ex13-184 | AACACTGCCAGATGTGTGAGTGT | Cpf1/Cas12f | 428. |
| Ex13-185 | TGACCCAATCAGTGGCCAGTGCC | Cpf1/Cas12f | 429. |
| Ex13-186 | TTACCTGGTTGACACTGATTACA | Cpf1/Cas12f | 430. |
| Ex13-187 | TTTCTTACCTGGTTGACACTGAT | Cpf1/Cas12f | 431. |

### AAV Delivery Systems

The methods include delivery of a CRISPR/Cas9 genome editing system, including a Cas9 nuclease and one or two guide RNAs, to a subject in need thereof. The delivery methods can include, e.g., viral delivery, preferably using an adeno-associated virus (AAV) vector that encodes the Cas9 and one or more guide RNA(s). AAV is a naturally occurring defective virus that requires another virus, such as an adenovirus or a herpes virus, as a helper virus for efficient replication and a productive life cycle. (For a review, see Muzyczka et al., Curr. Topics in Micro and Immunol. 158:97-129 (1992)). AAV vectors efficiently transduce various cell types and can produce long-term expression of transgenes in vivo. AAV vectors have been extensively used for gene augmentation or replacement and have shown therapeutic efficacy in a range of animal models as well as in the clinic; see, e.g., Mingozzi and High, Nature Reviews Genetics 12, 341-355 (2011); Deyle and Russell, Curr Opin Mol Ther. 2009 August; 11 (4): 442-447; Asokan et al., Mol Ther. 2012 April; 20 (4): 699-708. AAV vectors containing as little as 300 base pairs of AAV can be packaged and can produce recombinant protein expression. For example, AAV2, AAV5, AAV2/5, AAV2/8 and AAV2/7 vectors have been used to introduce DNA into photoreceptor cells (see, e.g., Pang et al., Vision Research 2008, 48 (3): 377-385; Khani et al., Invest Ophthalmol Vis Sci. 2007 September; 48 (9): 3954-61; Allocca et al., J. Virol. 2007 81 (20): 11372-11380). In some embodiments, the AAV vector can include (or include a sequence encoding) an AAV capsid polypeptide described in PCT/US2014/060163; for example, a virus particle comprising an AAV capsid polypeptide having an amino acid sequence selected from the group consisting of SEQ ID NOs: 1, 3, 5, 7, 9, 11, 13, 15, and 17 of PCT/US2014/060163, and a Cas9 sequence and guide RNA sequence as described herein. In some embodiments, the AAV capsid polypeptide is an Anc80 polypeptide, e.g., Anc80L27; Anc80L59; Anc80L60; Anc80L62; Anc80L65; Anc80L33; Anc80L36; or Anc80L44. In some embodiments, the AAV incorporates inverted terminal repeats (ITRs) derived from the AAV2 serotype. Exemplary left and right ITRs are presented in Table 6 of WO 2018/026976. It should be noted, however, that numerous modified versions of the AAV2 ITRs are used in the field, and the ITR sequences shown below are exemplary and are not intended to be limiting. Modifications of these sequences are known in the art, or will be evident to skilled artisans, and are thus included in the scope of this disclosure.

Cas9 expression can be driven by a promoter known in the art. In some embodiments, expression is driven by one of three promoters: cytomegalovirus (CMV), elongation factor-1 (EFS), or human g-protein receptor coupled kinase-1 (hGRK1), which is specifically expressed in retinal photoreceptor cells. Nucleotide sequences for each of these promoters are provided in Table 5 of WO 2018/026976. Modifications of these sequences may be possible or desirable in certain applications, and such modifications are within the scope of this disclosure.

Expression of the gRNAs in the AAV vector is driven by a promoter known in the art. In some embodiments, a polymerase III promoter, such as a human U6 promoter. An exemplary U6 promoter sequence is presented below:

(SEQ ID NO: 436)
AAGGTCGGGCAGGAAGAGGGCCTATTTCCCATGATTCCTTCATATTTGC
ATATACGATACAAGGCTGTTAGAGAGATAATTAGAATTAATTTGACTGT
AAACACAAAGATATTAGTACAAAATACGTGACGTAGAAAGTAATAATTT
CTTGGGTAGTTTGCAGTTTTAAAATTATGTTTTAAAATGGACTATCATA
TGCTTACCGTAACTTGAAAGTATTTCGATTTCTTGGCTTTATATATCTT
GTGGAAAGGACGAAACACC.

In some embodiments, the nucleic acid or AAV vector shares at least 80%, 85%, 90%, 95%, 96%, 97%, 98%, 99% or greater sequence identity with one of the nucleic acids or AAV vectors recited above

The AAV genomes described above can be packaged into AAV capsids (for example, AAV5 capsids), which capsids can be included in compositions (such as pharmaceutical compositions) and/or administered to subjects. An exemplary pharmaceutical composition comprising an AAV capsid according to this disclosure can include a pharmaceutically acceptable carrier such as balanced saline solution (BSS) and one or more surfactants (e.g., Tween 20) and/or a thermosensitive or reverse-thermosensitive polymer (e.g., pluronic). Other pharmaceutical formulation elements known in the art may also be suitable for use in the compositions described here.

Compositions comprising AAV vectors according to this disclosure can be administered to subjects by any suitable means, including without limitation injection, for example, subretinal injection or injection through the round window. The concentration of AAV vector within the composition is selected to ensure, among other things, that a sufficient AAV dose is administered to the retina or inner ear of the subject, taking account of dead volume within the injection apparatus and the relatively limited volume that can be safely administered. Suitable doses may include, for example, 1×10¹¹ viral genomes (vg)/mL, 2×10¹¹ viral genomes (vg)/mL, 3×10¹¹ viral genomes (vg)/mL, 4×10¹¹ viral genomes (vg)/mL, 5×10¹¹ viral genomes (vg)/mL, 6×10¹¹ viral genomes (vg)/mL, 7×10¹¹ viral genomes (vg)/mL, 8×10¹¹ viral genomes (vg)/mL, 9×10¹¹ viral genomes (vg)/mL, 1×10¹² vg/mL, 2×10¹² viral genomes (vg)/mL, 3×10¹² viral genomes (vg)/mL, 4×10¹² viral genomes (vg)/mL, 5×10¹² viral genomes (vg)/mL, 6×10¹² viral genomes (vg)/mL, 7×10¹² viral genomes (vg)/mL, 8×10¹² viral genomes (vg)/mL, 9×10¹² viral genomes (vg)/mL, 1×10¹³ vg/mL, 2×10¹³ viral genomes (vg)/mL, 3×10¹³ viral genomes (vg)/mL, 4×10¹³ viral genomes (vg)/mL, 5×10¹³ viral genomes (vg)/mL, 6×10¹³ viral genomes (vg)/mL, 7×10¹³ viral genomes (vg)/mL, 8×10¹³ viral genomes (vg)/mL, or 9×10¹³ viral genomes (vg)/mL. Any suitable volume of the composition may be delivered to the subretinal or cochlear space. In some instances, the volume is selected to form a bleb in the subretinal space, for example 1 microliter, 10 microliters, 50 microliters, 100 microliters, 150 microliters, 200 microliters, 250 microliters, 300 microliters, etc.

Any region of the retina may be targeted, though the fovea (which extends approximately 1 degree out from the center of the eye) may be preferred in certain instances due to its role in central visual acuity and the relatively high concentration of cone photoreceptors there relative to peripheral regions of the retina. Alternatively or additionally, injections may be targeted to parafoveal regions (extending between approximately 2 and 10 degrees off center), which are characterized by the presence of all three types of retinal photoreceptor cells. In addition, injections into the parafoveal region may be made at comparatively acute angles using needle paths that cross the midline of the retina. For instance, injection paths may extend from the nasal aspect of the sclera near the limbus through the vitreal chamber and into the parafoveal retina on the temporal side, from the temporal aspect of the sclera to the parafoveal retina on the nasal side, from a portion of the sclera located superior to the cornea to an inferior parafoveal position, and/or from an inferior portion of the sclera to a superior parafoveal position. The use of relatively small angles of injection relative to the retinal surface may advantageously reduce or limit the potential for spillover of vector from the bleb into the vitreous body and, consequently, reduce the loss of the vector during delivery. In other cases, the macula (inclusive of the fovea) can be targeted, and in other cases, additional retinal regions can be targeted, or can receive spillover doses.

For delivery to the inner ear, injection to the cochlear duct, which is filled with high potassium endolymph fluid, could provide direct access to hair cells. However, alterations to this delicate fluid environment may disrupt the endocochlear potential, heightening the risk for injection-related toxicity. The perilymph-filled spaces surrounding the cochlear duct, scala tympani and scala vestibuli, can be accessed from the middle ear, either through the oval or round window membrane (RWM). The RWM, which is the only non-bony opening into the inner ear, is relatively easily accessible in many animal models and administration of viral vector using this route is well tolerated. Administration through the oval window or across the tympanic membrane can also be used. See, e.g., WO2017100791 and U.S. Pat. No. 7,206,639.

For pre-clinical development purposes, systems, compositions, nucleotides and vectors according to this disclosure can be evaluated ex vivo using a human retinal explant system, or in vivo using an animal model such as a mouse, rabbit, pig, nonhuman primate, etc. Retinal explants are optionally maintained on a support matrix, and AAV vectors can be delivered by injection into the space between the photoreceptor layer and the support matrix, to mimic subretinal injection. Tissue for retinal explanation can be obtained from human or animal subjects, for example mouse.

Explants are particularly useful for studying the expression of gRNAs and/or Cas9 following viral transduction, and for studying genome editing over comparatively short intervals. These models also permit higher throughput than may be possible in animal models, and can be predictive of expression and genome editing in animal models and subjects. Small (mouse, rat) and large animal models (such as rabbit, pig, nonhuman primate) can be used for pharmacological and/or toxicological studies and for testing the systems, nucleotides, vectors and compositions of this disclosure under conditions and at volumes that approximate those that will be used in clinic. Because model systems are selected to recapitulate relevant aspects of human anatomy and/or physiology, the data obtained in these systems will generally (though not necessarily) be predictive of the behavior of AAV vectors and compositions according to this disclosure in human and animal subjects.

### EXAMPLES

The invention is further described in the following examples, which do not limit the scope of the invention described in the claims.

### Example 1

### Efficient Deletion of Mouse Ush2A Exon 12 Splicing Acceptor Using Paired-sgRNA in Mice Cells

Mouse organ of Corti HEI-OC1 cells were used as an in vitro cell line model to test the methods disclosed herein. HEI-OC1 cells are derived from mouse cochlea (Kalinec et al. (1999) Cell biology international 23 (3): 175-184; Kelley et al. (1993) Development 119 (4): 1041-1053). HEI-OC1 cells express USHERIN, making these cells particularly appropriate for these studies.

CRISPR/Cas9 genome editing technology was used to induce mouse exon 12 or human exon 13 skipping in Ush2a/USH2A transcripts. The LONZA 4D-Nucleofector™ was used to deliver Cas9/sgRNA protein complex in to the cells. Transfection programs were optimized following manufacturer's instruction (DS120, SG Cell Line 4D-Nucleofector™ X Kit). Purified sgRNA was incubated with Cas9 protein for 5 minutes before transfection. Media was replaced approximately 16 hours after nucleofection and cells were harvested for subsequent genomic DNA extraction after approximately 96 hours.

These results demonstrate that using CRISPR/Cas9 with a pair of sgRNAs, one targeting intron 11 and one targeting intron 12, can delete the full exon 12 from the mouse genome in HEI-OC1 cells. Different pairs of sgRNAs were screened, and one pair of sgRNAs was identified, sgL1/sgR1, which had 23.4% deletion efficiency (FIGS. 2A-C). Next, the genome editing strategy was redesigned, targeting the exon 12 splicing acceptor for removal, with this strategy referred to as an acceptor targeting paired-sgRNA strategy (FIG. 2E). The acceptor targeting paired-sgRNA strategy increased the genome editing efficiency, with the sgRNA pair sgL1 and sgK4 demonstrating an editing efficiency greater than 60% (FIG. 2D, 2F, 2G). RT-PCR analysis of the Ush2A transcripts showed that after genome editing the exon 12 skipping in-frame transcript were increased in OC1 cells (FIG. 2H, 2I). Exon 12 skipping was also observed in unedited HEI-OC1 cells, contrasted with human cells as spontaneous exon skipping has not been observed in human cells. These results demonstrate that the acceptor targeting paired-sgRNA strategy efficiently induces exon 12 skipping in mouse HEI-OC1 cells.

### Example 2

### Efficient Targeting at USH2A Exon 13 Loci Using Splicing Acceptor Targeting Strategy in Human Cells

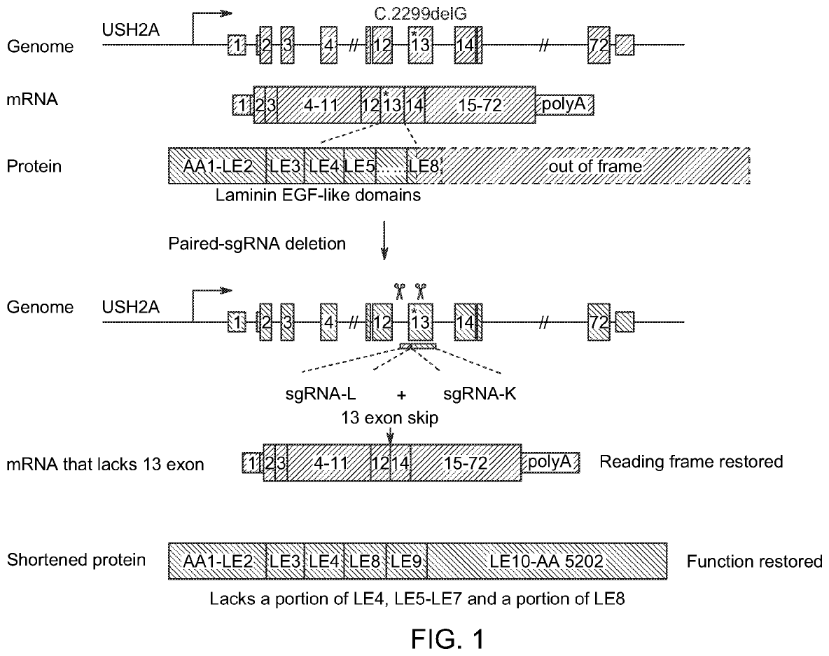

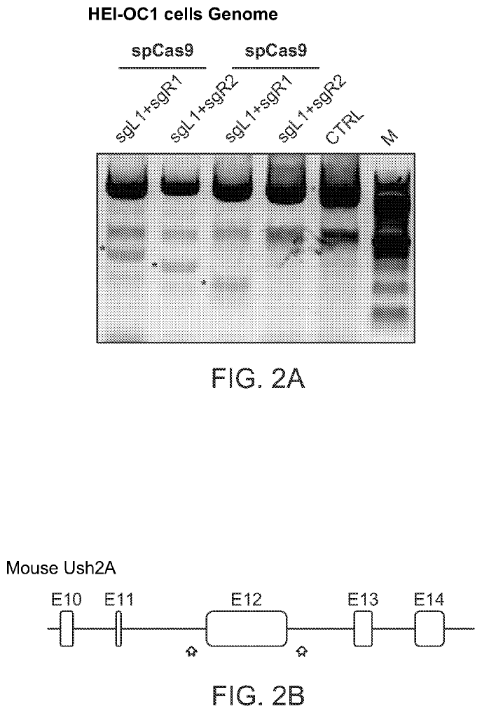

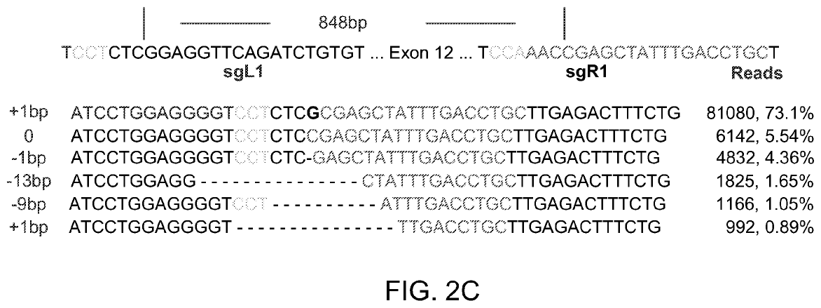

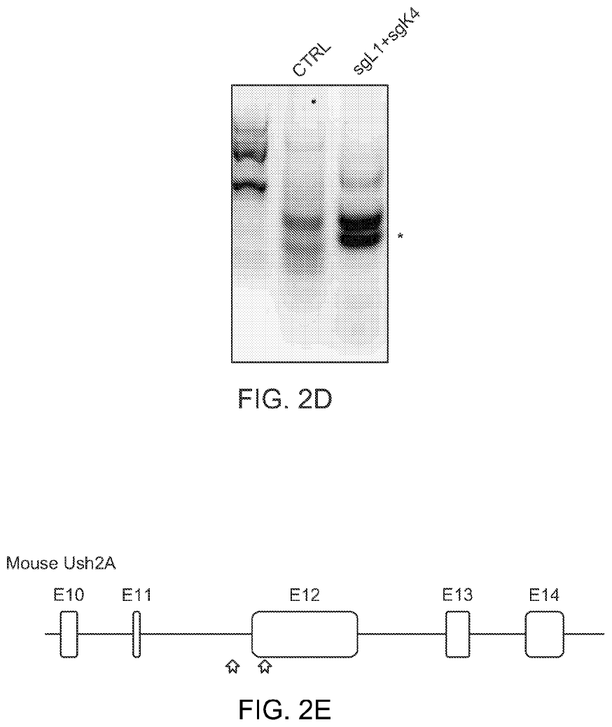

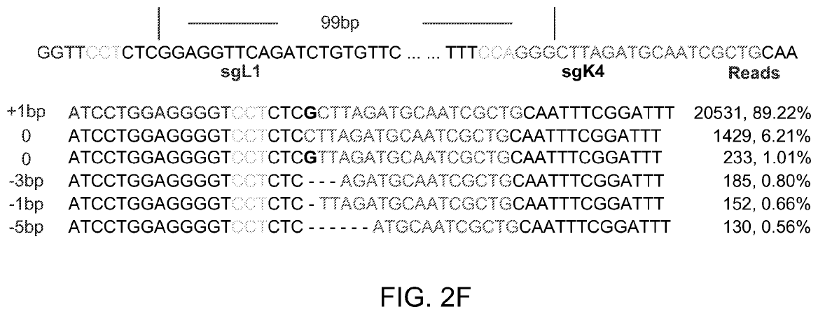

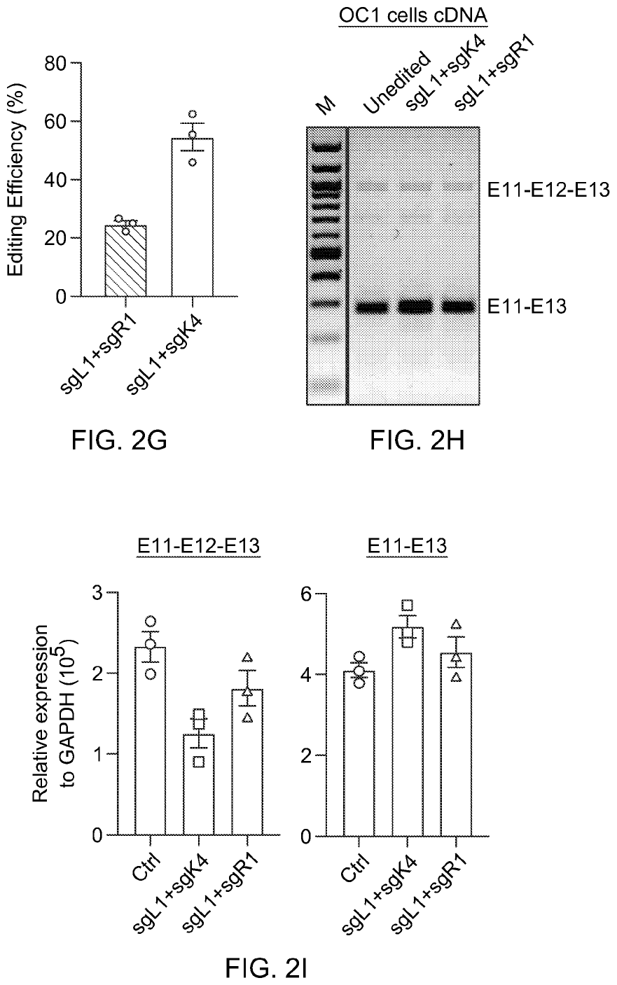

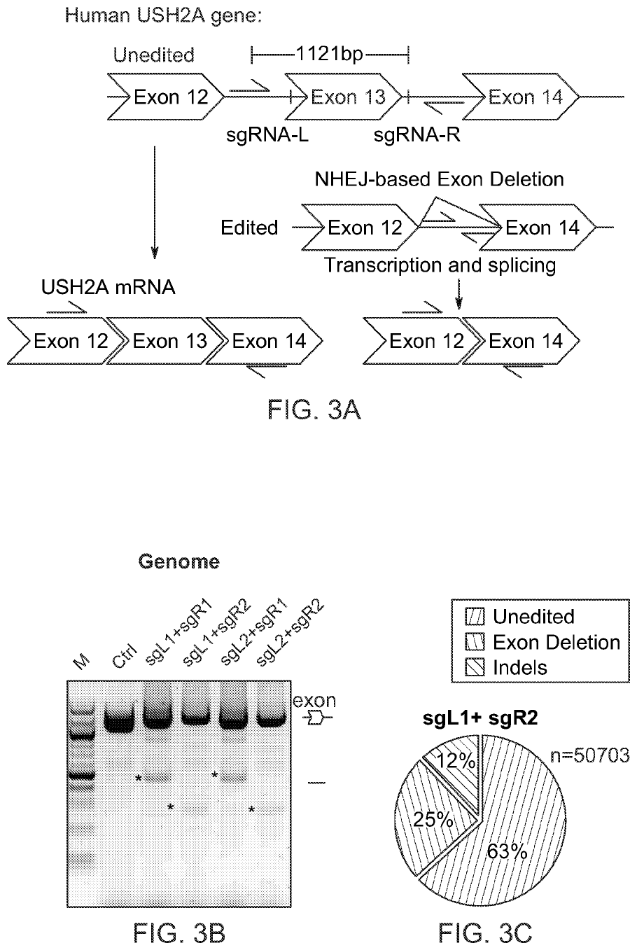

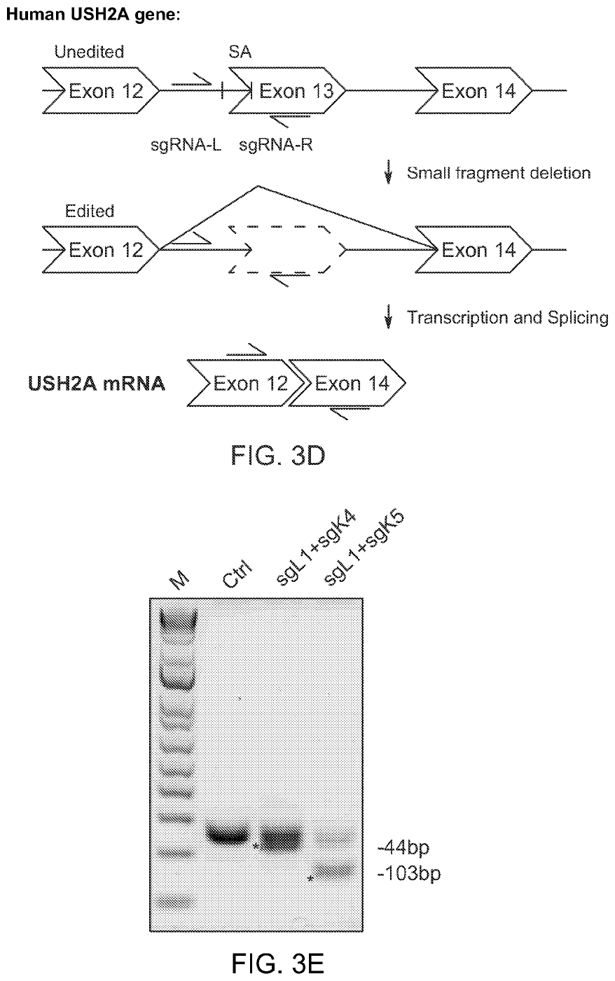

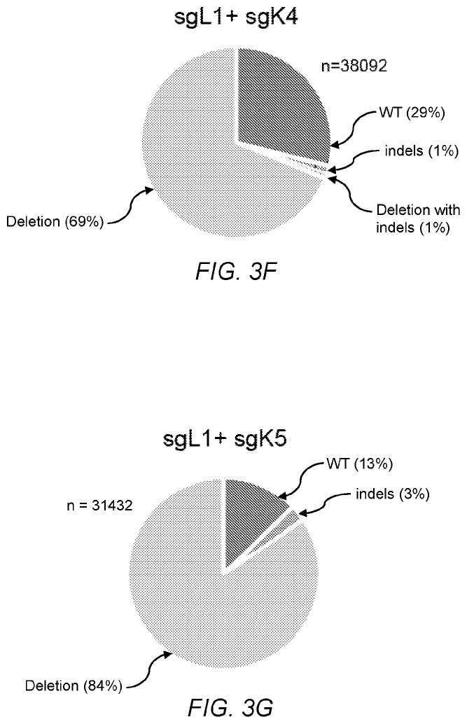

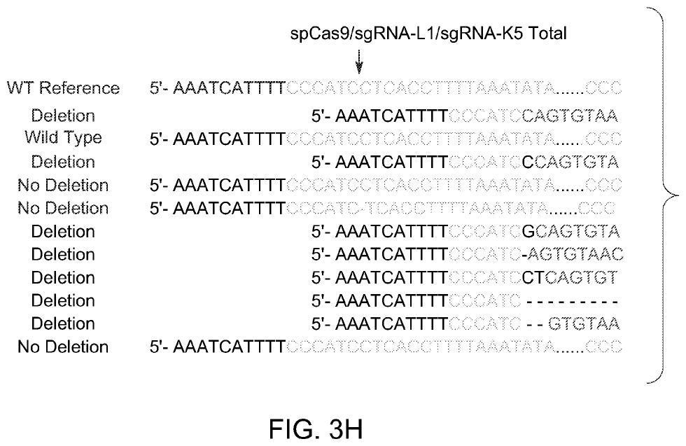

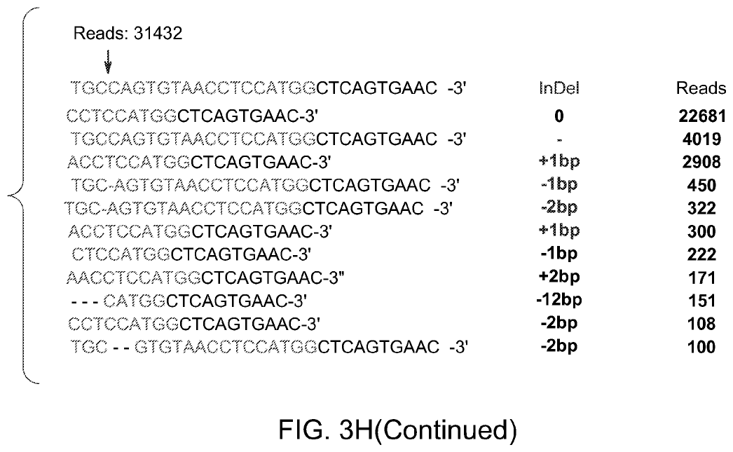

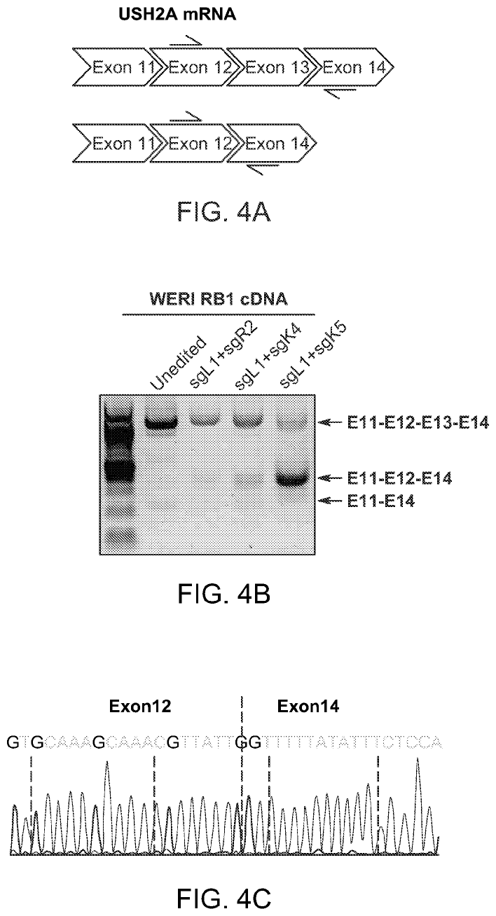

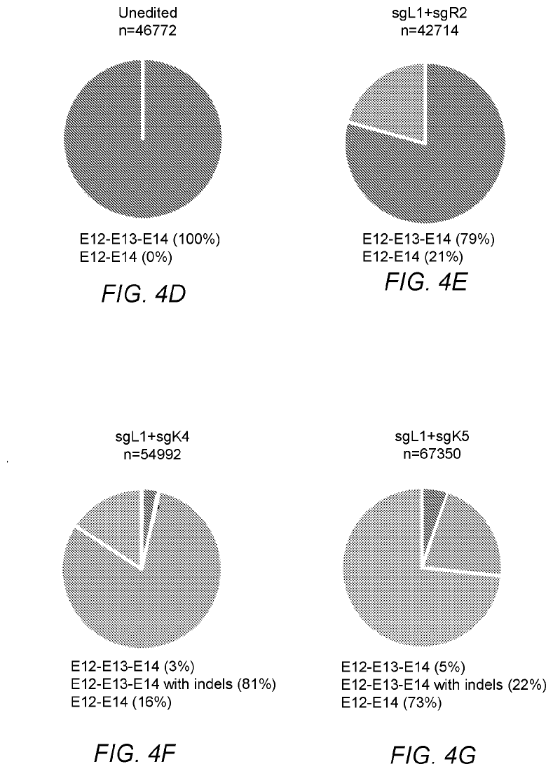

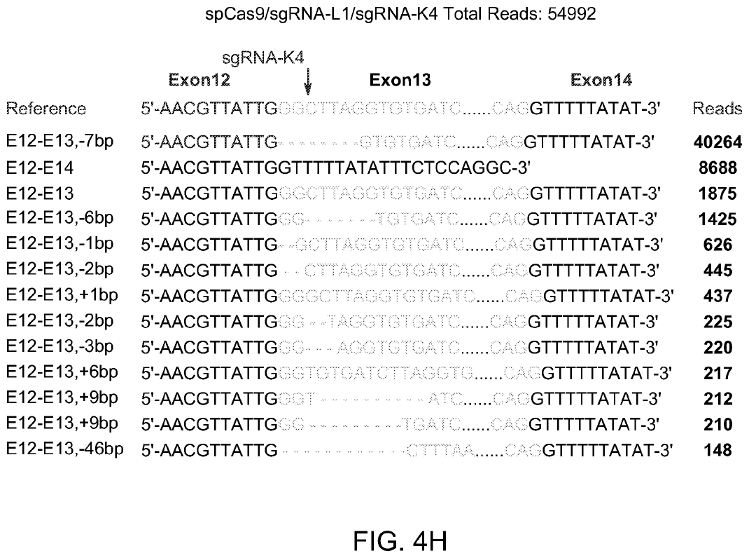

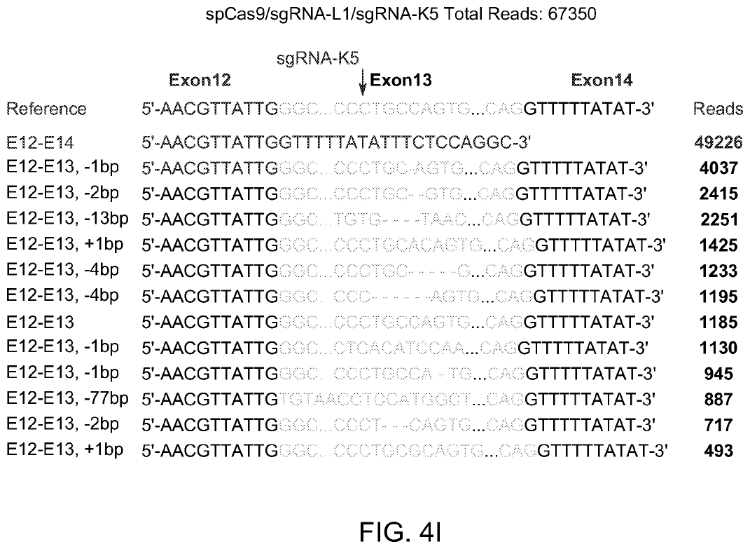

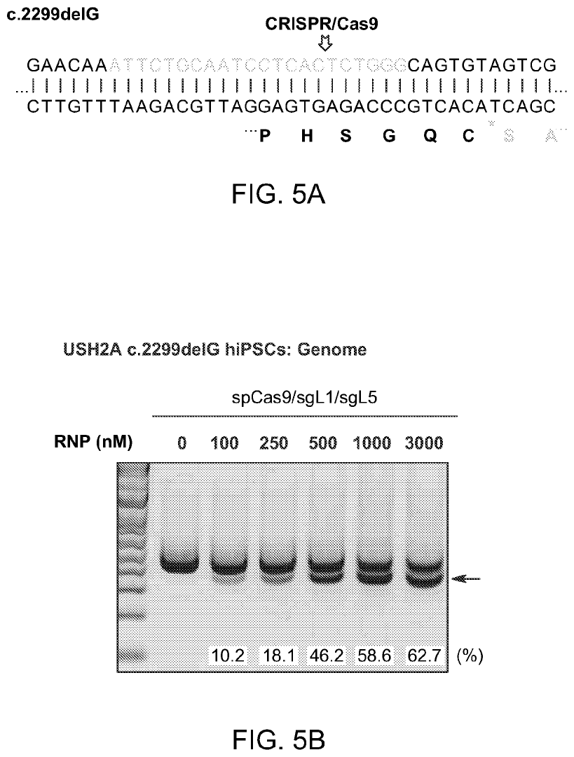

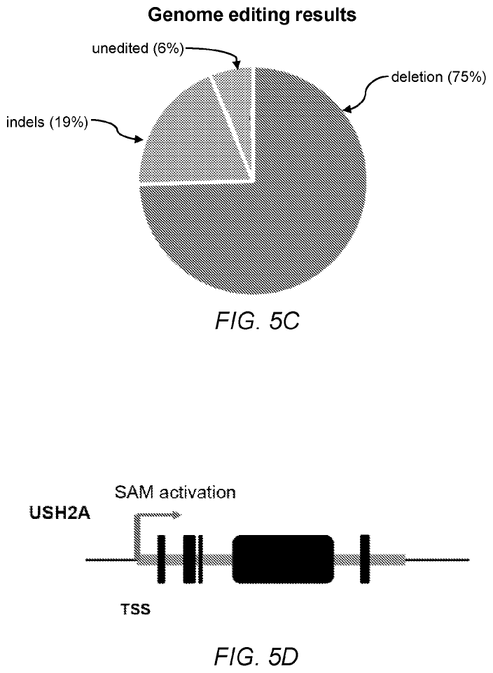

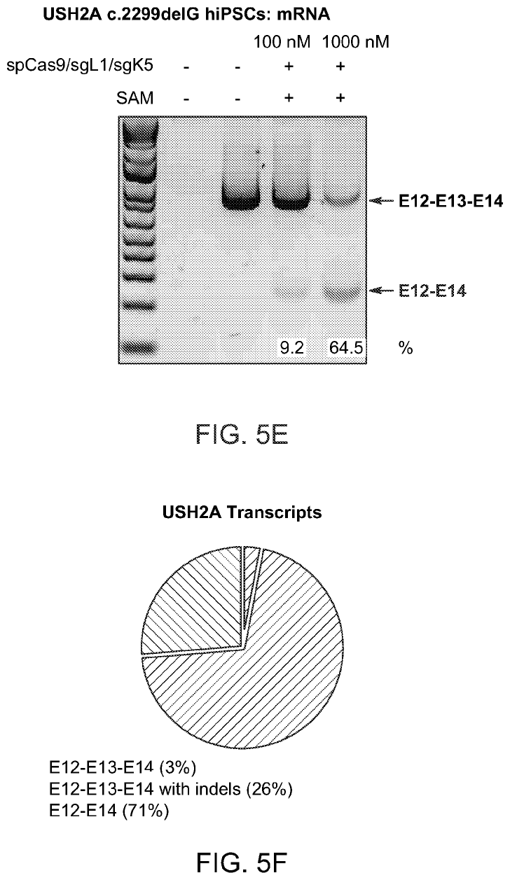

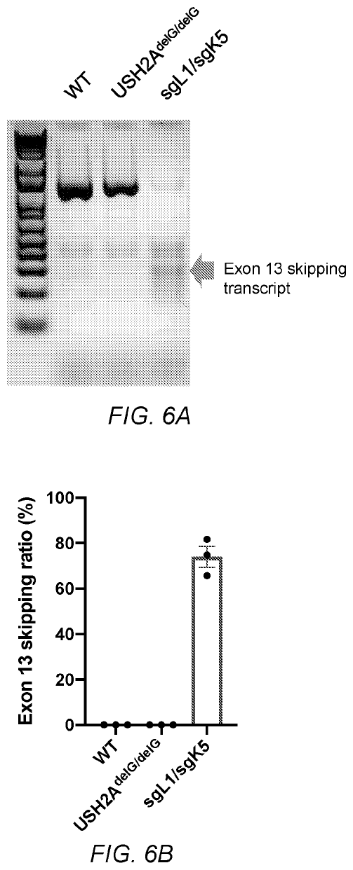

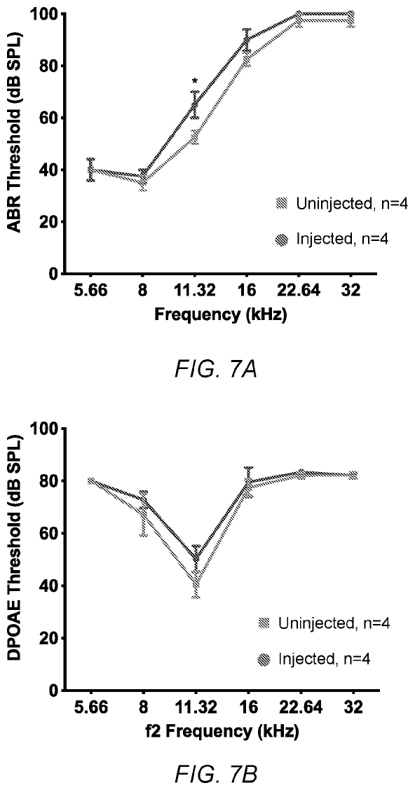

In order to evaluate the acceptor targeting paired-sgRNA strategy in human cells, a human retinoblastoma-derived cell line (WERI-RB1) was used, as WERI-RB1 cells express USHERIN. Multiple paired-sgRNA combinations were screened to test the deletion efficiency at human USH2A exon 13, with each pair of sgRNAs including one sgRNA targeting human USH2A intron 12 and the other targeting exon 13 (FIG. 1). The removal of the exon 13 splicing acceptor by paired sgRNAs should abolish the recognition of the remaining exon 13 as an exon, with the production of a USH2A transcript with exon 13 skipped (FIG. 3D). SpCas9 protein and paired sgRNAs were combined to form RNPs and delivered into the cells by nucleofection. 48 hours later, genomic DNA was extracted from the cells. Next, PCR and next generation sequencing (NGS) were performed to evaluate the deletion efficiency at the USH2A locus. NGS analysis showed that the acceptor targeting paired-sgRNA strategy resulted in deletion efficiency of 69% for sgL1/sgK4 pair (having target sequences corresponding to SEQ ID NO: 17 and SEQ ID NO: 313, respectively) and 84% for sgL1/sgK5 pair (having target sequences corresponding to SEQ ID NO: 17 and SEQ ID NO: 260, respectively) (FIG. 3E-h). In contrast, the full exon 13 deletion strategy only reached an efficiency of ˜25% in exon 13 skipping (FIG. 3A-C).

Using RT-PCR, the percentage of in-frame exon 13 skipping in USH2A transcripts was evaluated after genome editing. Two pairs of primers were designed to detect wild type USH2A transcripts (E12-E13-E14) and the exon 13 skipping transcripts (E12-E14) (FIG. 4A). 96 hours after nucleofection, total RNA was extracted from approximately 10⁶ cells using the miRNeasy Mini Kit (QIAGEN, 217004). Next, first-strand cDNA was produced using PrimeScript™ RT reagent Kit with gDNA Eraser (Takara) with random hexamers, following the manufacturer's instructions. PCR was performed and results indicated that sgL1/sgK5 generated the highest exon 13 skipping induction efficiency (FIG. 4B). Sanger sequencing of shorter fragment from DNA gels confirmed in-frame exon 13 skipping. NGS was performed on cDNA reverse transcribed from the USH2A transcripts in order to analyse different types of USH2A transcripts generated by genome editing. The result showed that sgL1/sgK5 combination yielded the highest efficiency of exon 13-skipped in-frame USH2A transcripts: 73% of all the USH2A transcripts were exon 13-skipped in-frame transcripts (FIG. 4D-G). The NGS reads of sgL1/sgK4 and sgL1/sgK5 are shown in FIGS. 4H, 4I. These data demonstrate that the acceptor targeting paired-sgRNA strategy mediated USH2A exon 13 skipping with the production of in-frame USH2A transcripts in human cells.

### Example 3

### Efficient Induction of USH2A Exon 13-Skipped In-Frame Transcripts from the USH2A Patient-Derived hiPSCs

In order to test the acceptor targeting paired-sgRNA strategy in human cells carrying an USH2A exon 13 mutation, human induced pluripotent stem cells (hiPSCs) derived from a female USH2 patient were obtained. The cells carried homozygous c.2299delG mutations (FIG. 5A). c.2299delG mutation is located in human USH2A exon 13 and is the most frequent pathogenic mutation observed in Usher syndrome patients. sgL1/sgK5 sgRNAs were used to induce USH2A exon 13 skipping in c.2299delG hiPSCs. Several Cas9/sgL1/sgK5 RNP dosages were tested using nucleofection, with the results indicating dose-dependent deletion efficiency with the highest efficiency obtained at a dosage above 1000 nM (FIG. 5B). NGS analysis of genomic DNA extracted from edited hiPSCs indicated that 75% of reads included skipped exon 13 (FIG. 5C).

Because USH2A is not expressed in hiPSCs, in order to analyse USH2A transcripts after editing, the synergistic activation mediator (SAM) system was used to activate USH2A expression (FIG. 5D) in hiPSCs. PB-SAM donor plasmid (addgene, 102559) with the sgRNAs to activate USH2A expression was co-transfected with PiggyBac transposon vector (PB210PA, System Biosciences) in the c.2299delG hiPSCs after genome editing with the Cas9/sgL1/sgK5 RNP. Cells were cultured and selected in growth medium containing 10 g/mL Blasticidin. Total mRNA was isolated, cRNA was reverse transcribed, and NGS and RT-PCR were performed in order to analyze expressed USH2A transcripts with exon 13 skipped. Gel imaging showed 1000 nM RNP delivery of a pair of sgRNAs sgL1/sgK5 induced efficient USH2A exon 13 skipping (FIG. 5E), and NGS demonstrated 73% of all USH2A transcripts included in-frame skipping of exon 13. Taken together, these data demonstrate that the acceptor targeting paired-sgRNA strategy using the Cas9/sgL1/sgK5 RNP shows therapeutic potential for treating disease associated with mutations of USH2A exon 13 mutation by efficiently inducing in-frame exon 13 skipping.

### Example 4

### Efficient Exon Skipping in Human USH2A Patient Inner Ear Organoids

Wild-type hiPSCs, USH2A^{delG/delG} hiPSCs, and genome-edited USH2A^{delG/delG} hiPSCs were differentiated into inner ear organoids, which model human cochlea development in vivo. Differentiation of each of the three types of hiPSCs generated hair cell-containing inner ear organoids. On day 50, organoid samples were collected, total mRNA was extracted, and cDNA was reversed transcribed. RT-PCR performed on the cDNAs showed that in organoids generated from wild-type hiPSCs and from USH2A^{delG/delG} hiPSCs, only full length USH2A transcripts comprising exons 12, 13, and 14 were detected. In contrast, as shown by the gel image of FIG. 6A, in organoids generated from hiPSCs edited with Cas9/sgL1/sgK5 RNP, efficient exon 13 skipping was detected. As shown in FIG. 6B, the exon 13-skipped transcript accounts for over 75% of USH2A transcripts detected in organoids generated from hiPSCs edited with Cas9/sgL1/sgK5 RNP.

### Example 5

### Exon 5 Skipping in Ush2a Gene Causes Hearing Loss

Using the paired sgRNA genome editing strategy, AAV vectors targeting Ush2a exon 5 and exon 12 (which is equivalent to human USH2A exon 13) in the mice genome were designed. The AAVs vectors were administered to an Usher syndrome mouse model harborying a heterozygous Ush2a mutation (Ush2a^{+/-}). The hearing test demonstrated that exon 5 skipping, but not exon 12 skipping, can lead to hearing loss in the mouse model. This study indicated that the requirement of exon 5, but not exon 12, for functional USHERIN protein in normal hearing in the Usher syndrome mouse model.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A nucleic acid comprising sequences encoding a Cas9 protein, and a first gRNA, and a second gRNA, wherein the target sequence of the first gRNA is any one of SEQ ID NOs: 2-244 and the target sequence of the second gRNA is any one of SEQ ID NOs: 245-431.

2. The nucleic acid of claim 1, wherein the target sequence of the first gRNA is SEQ ID NO: 17 and the target sequence of the second gRNA is SEQ ID NO: 313.

3. The nucleic acid of claim 1, wherein the target sequence of the first gRNA is SEQ ID NO: 17 and the target sequence of the second gRNA is SEQ ID NO: 260.

4. The nucleic acid of any of claim 1, wherein the nucleic acid encodes S. pyogenes Cas9 or S. aureus Cas9, optionally KKH SaCas9.

5. The nucleic acid of claim 1, wherein the Cas9 comprises a nuclear localization signal, e.g., a C-terminal nuclear localization signal and/or an N-terminal nuclear localization signal; and/or wherein the sequences encoding Cas9 comprises a polyadenylation signal.

6. The nucleic acid of claim 1, wherein the gRNA is a unimolecular S. pyogenes or S. aureus gRNA, or the corresponding two-part modular S. pyogenes or S. aureus gRNA.

7. The nucleic acid of one of claim 1, which comprises a viral delivery vector, preferably an adeno-associated virus (AAV) vector.

8. The nucleic acid of claim 7, wherein the viral delivery vector comprises a promoter for Cas9, preferably a CMV, EFS, U1A, or hGRK1 promoter.

9. The nucleic acid of claim 8, which comprises:
(i) a first guide RNA comprising a targeting domain sequence selected from any one of SEQ ID NOs: 2-244 and a second guide RNA comprising a targeting domain sequence selected from any one of SEQ ID NOs: 245-431;
(ii) a first and a second inverted terminal repeat sequence (ITR); and
(iii) a promoter for driving expression of the Cas9 selected from the group consisting of a CMV, an EFS, an U1A, or an hGRK1 promoter.

10. The nucleic acid of claim 1, for use in therapy.

11. The nucleic acid of claim 1, for use in preparation of a medicament.

12. The nucleic acid of claim 1, for use in a method of treating a subject who has a condition associated with a mutation in exon 13 of USH2A gene.

13. The nucleic acid for the use of claim 12, wherein the condition is Usher syndrome type 2 or autosomal recessive retinitis pigmentosa (arRP).

14. The nucleic acid for the use of claim 12, wherein the AAV vector is delivered to a retina of a subject by injection, such as by subretinal injection, or is delivered to the inner ear of a subject by injection, e.g., through the round window.

15. A composition comprising first ribonucleoprotein (RNP) complexes comprising a Cas9 protein and a first gRNA, and/or second RNP complexes comprising a Cas9 protein and a second gRNA, wherein the target sequence of the first gRNA is any one of SEQ ID NOs: 2-244 and the target sequence of the second gRNA is any one of SEQ ID NOs: 245-431.

16. The composition of claim 15, wherein the target sequence of the first gRNA is SEQ ID NO: 17 and the target sequence of the second gRNA is SEQ ID NO: 313.

17. The composition of claim 15, wherein the target sequence of the first gRNA is SEQ ID NO: 17 and the target sequence of the second gRNA is SEQ ID NO: 260.

18. The composition of claim 15, wherein the Cas9 is S. pyogenes Cas9 or S. aureus Cas9, optionally KKH SaCas9.

19. A method of deleting a sequence comprising an exon 13 splicing acceptor sequence from the USH2A gene in a cell, the deletion comprising part of intron 12 and part of exon 13, ranging from 6 bp to 2 KB, wherein the deletion induces human USH2A exon 13 skipping, the method comprising contacting the cell with the nucleic acid of claim 1.

20. A method of genome editing in human cells, the method comprising using CRISPR editing to form a first double strand break within intron 12 of the human USH2A gene and a second double strand break within exon 13 of the human USH2A gene and results in the removal of a fragment of genome DNA containing part of intron 12 and part of exon 13 of the USH2A gene on chromosome 1.

21. The method of claim 20, wherein the first double strand break is generated using a first gRNA having a target sequence of any one of SEQ ID NOs: 2-244 and the second double strand break is generated using a second gRNA having a target sequence of any one of SEQ ID NOs: 245-431.

22. The method of claim 21, wherein the target sequence of the first gRNA is SEQ ID NO: 17 and the target sequence of the second gRNA is SEQ ID NO: 313.

23. The method of claim 21, wherein the target sequence of the fir
st gRNA is SEQ ID NO: 17 and the target sequence of the second gRNA is SEQ ID NO: 260.

24. The method of claim 20, wherein the cell is in or from a subject who has a mutation in the USH2A gene.

25. The method of claim 20, wherein the cell is a cell of the eye or inner ear of a mammal.
